SEQUENCE LISTING (45 sequences)

SEQ ID NO: 1 - RNA, 19 nt, Artificial
  Synthetic
aguuggaaggaccacaugc

SEQ ID NO: 2 - RNA, 19 nt, Artificial
  Synthetic
gcaugugguccuuccaacu

SEQ ID NO: 3 - RNA, 23 nt, Artificial
  Synthetic
uuguccaggaaguccucaagucu

SEQ ID NO: 4 - RNA, 23 nt, Artificial
  Synthetic
agacuugaggacuuccuggacaa

SEQ ID NO: 5 - RNA, 23 nt, Artificial
  Synthetic
aucuugcugaaauucugauacug

SEQ ID NO: 6 - RNA, 23 nt, Artificial
  Synthetic
caguaucagaauuucagcaagau

SEQ ID NO: 7 - RNA, 23 nt, Artificial
  Synthetic
ucgaaguauuccgcguacgugau

SEQ ID NO: 8 - RNA, 23 nt, Artificial
  Synthetic
aucacguacgcggaauacuucga

SEQ ID NO: 9 - RNA, 19 nt, Artificial
  Synthetic
auaucugggcaaaugaugg

SEQ ID NO: 10 - RNA, 19 nt, Artificial
  Synthetic
ccaucauuugcccagauau

SEQ ID NO: 11 - RNA, 19 nt, Artificial
  Synthetic
uuccguucuagaguaucug

SEQ ID NO: 12 - RNA, 19 nt, Artificial
  Synthetic
cagauacucuagaacggaa

SEQ ID NO: 13 - RNA, 23 nt, Artificial
  Synthetic
acaaauucauuucucuugccccg

SEQ ID NO: 14 - RNA, 23 nt, Artificial
  Synthetic
cggggcaagagaaaugaauuugu

SEQ ID NO: 15 - RNA, 19 nt, Artificial
  Synthetic
gaauucacaguugcuucaa

SEQ ID NO: 16 - RNA, 19 nt, Artificial
  Synthetic
uugaagcaacugugaauuc

SEQ ID NO: 17 - RNA, 23 nt, Artificial
  Synthetic
auugauuguauucaucuuggcaa

SEQ ID NO: 18 - RNA, 23 nt, Artificial
  Synthetic
uugccaagaugaauacaaucaau

SEQ ID NO: 19 - DNA, 22 bp, Artificial
  Synthetic
atgagacagtgctggagggaga

SEQ ID NO: 20 - DNA, 23 bp, Artificial
  Synthetic
agcatcctgtttaaggacaccaa

SEQ ID NO: 21 - DNA, 29 bp, Artificial
  Synthetic
agccttatgagagaccatcatttgcccag

SEQ ID NO: 22 - DNA, 21 bp, Artificial
  Synthetic
gggaacgagagccacagagac

SEQ ID NO: 23 - DNA, 21 bp, Artificial
  Synthetic
cattaagggagccttccgttc

SEQ ID NO: 24 - DNA, 28 bp, Artificial
  Synthetic
cggaaggtcgaccctaatctcatggaaa

SEQ ID NO: 25 - DNA, 22 bp, Artificial
  Synthetic
tcaatggagaaacagagccaca

SEQ ID NO: 26 - DNA, 22 bp, Artificial
  Synthetic
gaatccataggcgagatcaagg

SEQ ID NO: 27 - DNA, 28 bp, Artificial
  Synthetic
tggtctctctggttgtgaatgtcccacc

SEQ ID NO: 28 - DNA, 20 bp, Artificial
  Synthetic
ccgtctggctcattctgtga

SEQ ID NO: 29 - DNA, 22 bp, Artificial
  Synthetic
gggagattgcgggtttataatg

SEQ ID NO: 30 - DNA, 31 bp, Artificial
  Synthetic
tggcttatcttcacacagaattaccacgagg

SEQ ID NO: 31 - DNA, 22 bp, Artificial
  Synthetic
gaacgaggacagcaacttcacc

SEQ ID NO: 32 - DNA, 22 bp, Artificial
  Synthetic
ctcccgattaaactgcccatac

SEQ ID NO: 33 - DNA, 28 bp, Artificial
  Synthetic
tcataaacaaccatgacaacaccgccaa

SEQ ID NO: 34 - DNA, 22 bp, Artificial
  Synthetic
caccgccaaatttaactgcaga

SEQ ID NO: 35 - DNA, 24 bp, Artificial
  Synthetic
aagggtttgataagttctagctgt

SEQ ID NO: 36 - DNA, 31 bp, Artificial
  Synthetic
tgcacagtatccttttgaagaccataaccca

SEQ ID NO: 37 - DNA, 22 bp, Artificial
  Synthetic
gtttgagaccttcaacacccca

SEQ ID NO: 38 - DNA, 21 bp, Artificial
  Synthetic
gaccagaggcatacagggaca

SEQ ID NO: 39 - DNA, 26 bp, Artificial
  Synthetic
ccatgtacgtagccatccaggctgtg

SEQ ID NO: 40 - DNA, 21 bp, Artificial
  Synthetic
gaggctgatgctggtgctagt

SEQ ID NO: 41 - DNA, 22 bp, Artificial
  Synthetic
cagcaaacactcaggcctaacc

SEQ ID NO: 42 - DNA, 27 bp, Artificial
  Synthetic
ctgctccctgcttctagcccagtctga

SEQ ID NO: 43 - DNA, 22 bp, Artificial
  Synthetic
caaaggattcggacaatgacaa

SEQ ID NO: 44 - DNA, 21 bp, Artificial
  Synthetic
cattcaagttggaaggaccac

SEQ ID NO: 45 - DNA, 28 bp, Artificial
  Synthetic
tcccagatgctctcaggaggctggtggt

(19) United States
(12) Patent Application Publication
(10) Pub. No.: US 2019/0038557 A1
(43) Pub. Date: Feb. 7, 2019

(54) MEANS FOR LUNG SPECIFIC DELIVERY

(71) Applicant: Silence Therapeutics GMBH, Berlin (DE)

(72) Inventors: Oliver KEIL, GLIENICKE (DE); Jörg KAUFMANN, Berlin (DE); Volker FEHRING, Berlin (DE); Ute SCHAEPER, Berlin (DE)

(21) Appl. No.: 15/897,069

(22) Filed: Feb. 14, 2018

Related U.S. Application Data
(63) Continuation of application No. 15/101,426, filed on Jun. 3, 2016, now abandoned.

(30) Foreign Application Priority Data
Dec. 5, 2013 (EP) 13005672.4

(51) Int. Cl. A61K 9/127; A61K 47/26; C12N 15/113; A61K 31/713

(52) U.S. Cl. CPC A61K 9/1272; A61K 47/26; C12N 15/1136; C12N 2320/32; A61K 9/1271; C12N 2310/343; A61K 31/713; C12N 2310/321; C12N 2310/14

(57) ABSTRACT

The present invention is related to composition comprising a lipid composition, wherein the lipid composition consists of a cationic lipid of formula (I) wherein n is any one of 1, 2, 3, and 4, wherein m is any one of 1, 2 and 3, Y′ is an anion, wherein each of R1 and R2 is individually and independently selected from the group consisting of linear C12-C18 alkyl and linear C12-C18 alkenyl; a sterol compound, wherein the sterol compound is selected from the group consisting of cholesterol and stigmasterol; and a PEGylated lipid, wherein the PEGylated lipid comprises a PEG moiety and wherein the PEGylated lipid is selected from the group consisting of a PEGylated phosphoethanolamine of formula (II) wherein each of R3 and R4 is individually and independently linear C13-C17 alkyl, and p is any integer from 15 to 130; a PEGylated ceramide of formula (III) wherein R5 is linear C7-C15 alkyl, and q is any integer from 15 to 130; and a PEGylated diacylglycerol of formula (IV) wherein each of R6 and R7 is individually and independently linear C11-C17 alkyl, and r is any integer from 15 to 130.

### CROSS REFERENCE TO RELATED APPLICATIONS

This application is a continuation application of U.S. patent application Ser. No. 15/101,426, filed Jun. 3, 2016, which is a National Phase filing under 35 U.S.C. § 371 of International Application No. PCT/EP2014/003274, filed Dec. 5, 2014, which claims priority to European Application No.: 13005672.4, filed Dec. 5, 2013, the disclosures of each of which are incorporated by reference herein in their entireties.

### SUBMISSION OF SEQUENCE LISTING ON ASCII TEXT FILE

The content of the following submission on ASCII text file is incorporated herein by reference in its entirety; a computer readable form (CRF) of the Sequence Listing (file name: 680132000201SEQLIST.TXT, date recorded: Oct. 10, 2018, size: 8 KB).

The present invention is related to a composition comprising a lipid composition; the composition comprising a lipid composition for use in a method for the treatment of a disease; use of the composition comprising a lipid composition for the manufacture of a medicament for the treatment and/or prevention of a disease; a pharmaceutical composition comprising composition comprising a lipid composition; use of the composition comprising a lipid composition as a transferring agent; a kit comprising the composition comprising a lipid composition; a method for transferring a biologically active compound or a pharmaceutically active compound into a cell or across a membrane of a cell, wherein the method comprises contacting the cell or the membrane of a cell with the composition comprising a lipid composition; a method for the treatment and/or prevention of a disease, wherein the method comprises administering to a subject in need thereof an effective amount of the composition comprising a lipid composition.

Both molecular biology as well as molecular medicine heavily rely on the introduction of biologically active compounds into cells. Such biologically active compounds typically comprise, among others, DNA, RNA as well as peptides and proteins, respectively. The barrier which has to be overcome is typically a lipid bilayer which has a negatively charged outer surface. In the art, a number of technologies have been developed to penetrate the cellular membrane and to thus introduce the biologically active compounds. Some methods conceived for laboratory use, however, cannot be used in the medical field and are more particularly not suitable for drug delivery. For example, electroporation and ballistic methods known in the art, would, if at all, only allow a local delivery of biologically active compounds. Apart from said lipid bilayer cellular membranes also comprise transporter systems. Accordingly, efforts were undertaken to use this kind of transporter systems in order to transfer the biologically active compounds across the cell membrane. However, due to the specificity or cross-reactivity of such transporter systems, their use is not a generally applicable method.

A more generally applicable approach described in the art for transferring biologically active compounds into cells, is the use of viral vectors. However, viral vectors can be used only for transferring genes efficiently into some cell types; but they cannot be used to introduce chemically synthesised molecules into the cells.

An alternative approach was the use of so called liposomes (Bangham, J. Mol. Biol, 13, 238-252). Liposomes are vesicles which are generated upon association of amphiphilic lipids in water. Liposomes typically comprise concentrically arranged bilayers of phospholipids. Depending on the number of layers liposomes can be categorised as small unilamelar vesicles, multilamelar vesicles and large multilamelar vesicles. Liposomes have proven to be effective delivery agents as they allow incorporating hydrophilic compounds into the aqueous intermediate layers, whereas hydrophobic compounds are incorporated into the lipid layers, it is well known in the art that both the composition of the lipid formulation as well as its method of preparation have an effect on the structure and size of the resultant lipid aggregates and thus on the liposomes. Liposomes are also known to incorporate cationic lipids.

Cationic lipids have, apart from being components of liposomes, also attracted considerable attention as they may as such be used for cellular delivery of biopolymers. Using cationic lipids any anionic compound can be encapsulated essentially in a quantitative manner due to electrostatic interaction. In addition, it is believed that the cationic lipids interact with the negatively charged cell membranes initiating cellular membrane transport. It has been found that the use of a liposomal formulation containing cationic lipids or the use of cationic lipids as such together with a biologically active compound requires a heuristic approach as each formulation is of limited use because it typically can deliver plasmids into some but not all cell types, usually in the absence of serum.

Charge and/or mass ratios of lipids and the biologically active compounds to be transported by them have turned out to be a crucial factor in the delivery of different types of said biologically active compounds. For example, it has been shown that lipid formulations suitable for plasmid delivery comprising 5,000 to 10,000 bases in size, are generally not effective for the delivery of oligonucleotides such as siRNA molecules, synthetic ribozymes or antisense molecules typically comprising about 10 to about 50 bases. In addition, it has recently been indicated that optimal delivery conditions for antisense oligonucleotides and ribozymes are different, even in the same cell type.

U.S. Pat. No. 6,395,713 discloses cationic lipid based compositions whereby the cationic lipid consist of a lipophilic group, a linker and a head group and the use of such compositions for transferring biologically active compounds into a cell.

International patent application WO 2005/105152 discloses another cationic lipid based composition which proved to be particularly effective in the delivery of functional nucleic acid molecule such as siRNA molecules.

Depending on the disease to be treated and the drug to be delivered, there is a need for delivering the drug to specific organs or specific cell types. One such specific organ is lung and one such specific cell type is pulmonary endothelial cell. The targeting of lung and pulmonary endothelial cell is, for example, advantageous in the delivery of a drug for the treatment of a disease such as acute lung injury, acute respiratory distress syndrome, lung cancer, pulmonary metastasis, pulmonary hypertension and pulmonary artery hypertension.

A problem underlying the present invention is the provision of a means which is capable of delivering an agent, preferably a therapeutically active agent, more preferably a drug, to lung. A further problem underlying the present invention is the provision of a means which is capable of delivering an agent, preferably a therapeutically active agent, more preferably a drug, to lung tissue. A still further problem underlying the present invention is the provision of a means which is capable of delivering an agent, preferably a therapeutically active agent, more preferably a drug, to a pulmonary endothelial cell.

Another problem underlying the present invention is the provision of a means for the treatment of a lung disease, preferably a lung disease which is selected from the group comprising acute lung injury, acute respiratory distress syndrome, lung cancer, pulmonary metastasis, pulmonary hypertension and pulmonary artery hypertension.

Another problem underlying the present invention is the provision of a delivery vehicle as part of a means for the treatment of a lung disease, preferably a lung disease which is selected from the group comprising aerate lung injury, acute respiratory distress syndrome, lung cancer, pulmonary metastasis, pulmonary hypertension and pulmonary artery hypertension.

Another problem underlying the present invention is the provision of a pharmaceutical composition. Preferable, the pharmaceutical is suitable for the delivery of an agent, preferably a therapeutically active agent, more preferably a drug, to lung. A further problem underlying the present invention is the provision of a pharmaceutical composition suitable for the delivery of an agent, preferably a therapeutically active agent, more preferably a drug, to lung tissue. A still further problem underlying the present invention is the provision of a pharmaceutical composition suitable tor the delivery of an agent, preferably a therapeutically active agent, more preferably a drug, to a pulmonary endothelial cell.

Another problem underlying the present invention is the provision of a means which can be used in the manufacture of a medicament, whereby the medicament is suitable for or is for use in the treatment of lung disease, preferably a lung disease which is selected from the group comprising acute lung injury, acute respiratory distress syndrome, lung cancer, pulmonary metastasis, pulmonary hypertension and pulmonary artery hypertension.

Another problem underlying the present invention is the provision of a method and/or prevention for the treatment of a disease, wherein the method comprises administering to a subject in need thereof an effective amount a composition comprising a therapeutically or pharmaceutical active agent, preferably a drug. A further problem underlying the present invention is the provision of a method for the treatment and/or prevention of a lung disease, wherein the method comprises administering to a subject in need thereof an effective amount a composition comprising a therapeutically or pharmaceutical active agent, preferably a drug. A still further problem underlying the present invention is the provision of a method for the treatment and/or prevention of a disease, preferably a lung disease, whereby the treatment comprises delivering a therapeutically or pharmaceutical active agent to the lung, preferably to a pulmonary endothelial cell.

Another problem underlying the present invention is the provision of a transferring agent. Preferably the transferring agent is capable of transferring a biologically active agent, a therapeutically active agent and/or or pharmaceutically active agent into a cell or across a membrane of a cell, whereby preferably such cell is a pulmonary endothelial cell.

Another problem underlying the present invention is the provision of a method for transferring a biologically active, a therapeutically active agent and/or a pharmaceutically active agent into a cell or across a membrane of a cell, whereby preferably such cell is a pulmonary endothelial cell.

Another problem underlying the present invention is the provision of a kit. Preferably, the kit is suitable (a) for use in a method for the treatment and/or prevention of a disease, preferably a lung disease, (b) for use in a method of transferring a biologically active agent, a therapeutically active agent and/or or pharmaceutically active agent into a cell or across a membrane of a cell, whereby preferably such cell is a pulmonary endothelial cell, and/or (c) for use in the manufacture of a medicament, preferably a medicament for the treatment and/or prevention of a disease, more preferably a lung disease and most preferably a lung disease selected from the group comprising acute lung injury, acute respiratory distress syndrome, lung cancer, pulmonary metastasis, pulmonary hypertension and pulmonary artery hypertension.

These and other problems are solved by the subject matter of the attached dependent claims. Preferred embodiments may be taken from the attached dependent claims. These and other problems are also solved by the following embodiments.

### EMBODIMENT 1

A Composition comprising a lipid composition, wherein the lipid composition consists of
a cationic lipid of formula (I)

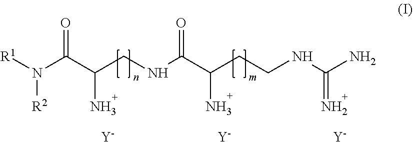

wherein n is any one of 1, 2, 3, and 4,
wherein m is any one of 1, 2 and 3,
Y⁻ is an anion,
wherein each of R1 and R2 is individually and independently selected from the group consisting of linear C12-C18 alkyl and linear C12-C18 alkenyl;
a sterol compound, wherein the sterol compound is selected from the group consisting of cholesterol and stigmasterol; and
a PEGylated lipid, wherein the PEGylated lipid comprises a PEG moiety and wherein the PEGylated lipid is selected from the group consisting of
   a PEGylated phosphoethanolamine of formula (II)

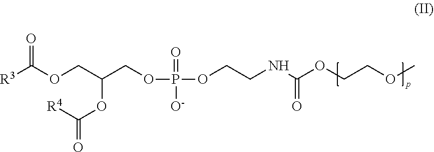

wherein each of R3 and R4 is individually and independently linear C13-C17 alkyl, and
p is any integer from 15 to 130;
a PEGylated ceramide of formula (III)

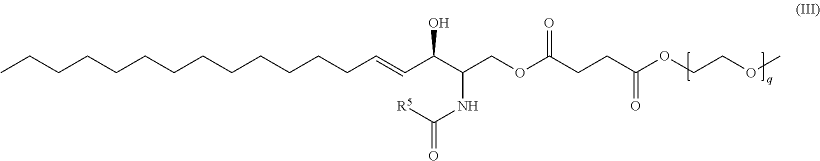

wherein R5 is linear C7-C15 alkyl, and
q is any integer from 15 to 130; and
a PEGylated diacylglycerol of formula (IV)

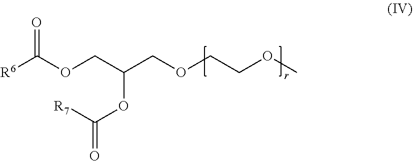

wherein each of R6 and R7 is individually and independently linear C11-C17 alkyl, and r is any integer from 15 to 130.

### EMBODIMENT 2

The composition of embodiment 1, wherein R1 and R2 are different from each other.

### EMBODIMENT 3

The composition of embodiment 1, wherein R1 and R2 are the same.

### EMBODIMENT 4

The composition of any one of embodiments 1 to 3, wherein each of R1 and R2 is individually and independently selected from the group consisting of C12 alkyl, C14alkyl, C16 alkyl, C18 alkyl, C12 alkenyl, C14 alkenyl, C16 alkenyl and C18 alkenyl.

### EMBODIMENT 5

The composition of embodiment 4, wherein each of C12 alkenyl, C14 alkenyl, C16 alkenyl and C18 alkenyl comprises one or two double bonds.

### EMBODIMENT 6

The composition of embodiment 5, wherein C18 alkenyl is C18 alkenyl with one double bond between C9 and C10, preferably cis-9-octadecyl].

### EMBODIMENT 7

The composition of any one of embodiments 1 to 6, wherein R1 and R2 are different and R1 is palmityl and R2 is oleyl.

### EMBODIMENT 8

The composition of any one of embodiments 1 to 6, wherein R1 and R2 are different and wherein R1 is lauryl and R2 is myristyl.

### EMBODIMENT 9

The composition of any one of embodiments 1 to 8, wherein the cationic lipid is a compound of formula (Ia)

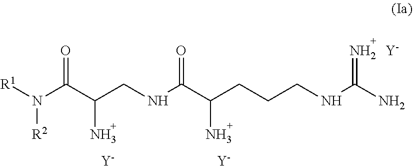

### EMBODIMENT 10

The composition of any one of embodiments 1 to 9, wherein Y⁻ is selected from the group comprising halogenids, acetate and trifluoroacetate.

### EMBODIMENT 11

The composition of embodiment 10, wherein Y⁻ is Cl⁻.

EMBODIMENT 12

The composition of any one of embodiments 1 to 11, wherein the cationic lipid is β-arginyl-2,3-diamino propionic acid-N-palmityl-N-oleyl-amide trihydrochloride of formula (Ib):

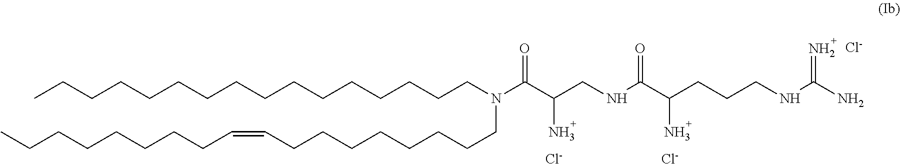 [SMILES: [Cl-] ; CCCCCCCC/C=C\CCCCCCCCN(CCCCCCCCCCCCCCCC)C(=O)C(N)CNC(=O)C(N)CCCNC(=N)N ; [Cl-] ; [Cl-]]

### EMBODIMENT 13

The composition of any one of embodiments 1 to 11, wherein the cationic lipid is β-arginyl-2,3-diamino propionic acid-N-lauryl-N-myristyl-amide trihydrochloride of formula (Ie):

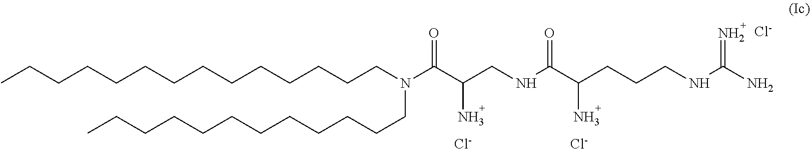 [SMILES: [Cl-] ; CCCCCCCCCCCCCCN(CCCCCCCCCCCC)C(=O)C(N)CNC(=O)C(N)CCCNC(=N)N ; [Cl-] ; [Cl-]]

### EMBODIMENT 14

The composition of any one of embodiments 1 to 11 wherein the cationic lipid is ϵ-arginyl-lysine-N-lauryl-N-myristyl-amide trihydrochloride of formula (Id):

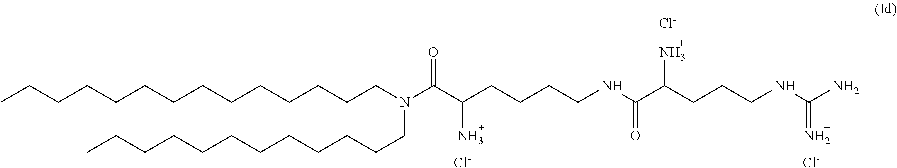 [SMILES: [Cl-] ; CCCCCCCCCCCCCCN(CCCCCCCCCCCC)C(=O)C(N)CCCCNC(=O)C(N)CCCNC(=N)N ; [Cl-] ; [Cl-]]

### EMBODIMENT 15

The composition of any one of embodiments 1 to 14, wherein the sterol compound is cholesterol.

### EMBODIMENT 16

The composition of any one of embodiments 12 to 14, preferably embodiment 14, wherein the sterol compound is cholesterol.

### EMBODIMENT 17

The compound of any one of embodiments 1 to 14, wherein the sterol compound is stigmasterin.

### EMBODIMENT 18

The composition of any one of embodiments 12 to 14, preferably embodiment 14, wherein the sterol compound is stigmasterin.

### EMBODIMENT 19

The composition of any one of embodiments 1 to 18, preferably any one of embodiments 12 to 14, more preferably any one of embodiments 16 and 18, wherein the PEG moiety of the PEGylated lipid has a molecular weight from about 800 to about 5000 Da.

### EMBODIMENT 20

The composition of embodiment 19, wherein the molecular weight of the PEG moiety of the PEGylated lipid is about 800 Da.

### EMBODIMENT 21

The composition of embodiment 19, wherein, the molecular weight of the PEG moiety of the PEGylated lipid is about 2000 Da.

### EMBODIMENT 22

The composition of embodiment 19, wherein the molecular weight of the PEG moiety of the PEGylated lipid is about 5000 Da.

### EMBODIMENT 23

The composition of any one of embodiments 1 to 22, preferably any one of embodiments 19 to 22, wherein the PEGylated lipid is a PEGylated phosphoethanolamine of formula (II), wherein each of R3 and R4 is individually and independently linear C13-C17 alkyl, and

p is any integer from 18, 19 or 20, or from 44, 45 or 46 or from 113, 114 or 115,

### EMBODIMENT 24

The composition of embodiment 23, wherein, R3 and R4 are the same.

### EMBODIMENT 25

The composition of embodiment 23, wherein R3 and R4 are different.

### EMBODIMENT 26

The composition of any one of embodiments 23 and 25, wherein each of R3 and R4 is individually and independently selected from the group consisting of C13 alkyl, OS alkyl and C17 alkyl.

### EMBODIMENT 27

The composition of any one of embodiments 1 to 26, preferably one of embodiments 12 to 14, more preferably any one of embodiments 14 and 16, wherein the PEGylated phosphoethanolamine of formula (II) is

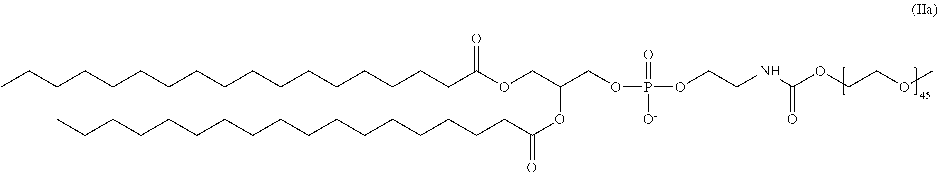

### EMBODIMENT 28

The composition of any one of embodiments 1 to 26, preferably one of embodiments 12 to 14, more preferably any one of embodiments 16 and 18, wherein the PEGylated phosphoethanolamine of formula (II) is

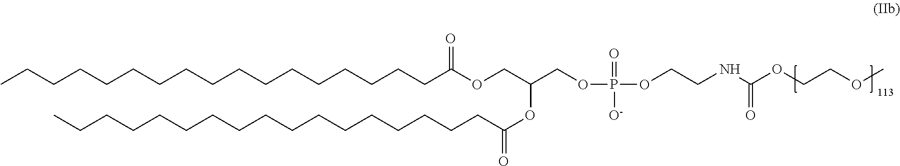

### EMBODIMENT 29

The composition of any one of embodiments 1 to 22, preferably any one of embodiments 19 to 22, wherein the PEGylated lipid is a PEGylated ceramide of formula (III), wherein R5 is linear C7-C15 alkyl, and

q is any integer from 18, 19 or 20, or from 44, 45 or 46 or from 113, 114 or 115.

### EMBODIMENT 30

The composition of embodiment 29, wherein R5 is linear C alkyl.

### EMBODIMENT 31

The composition of embodiment 30, wherein R5 is linear C15 alkyl.

### EMBODIMENT 32

The composition of any one of embodiments 1 to 22 and 29 to 31, preferably one of embodiments 12 to 14, more preferably any one of embodiments 16 and 18, wherein the PEGylated ceramide of formula (III) is

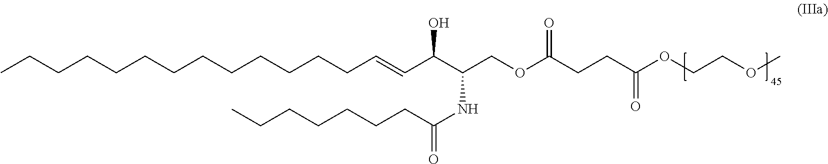

### EMBODIMENT 33

The composition of any one of embodiments 1 to 22 and 29 to 31, preferably one of embodiments 12 to 14, more preferably any one of embodiments 16 and 18, wherein the PEGylated ceramide of formula (III) is

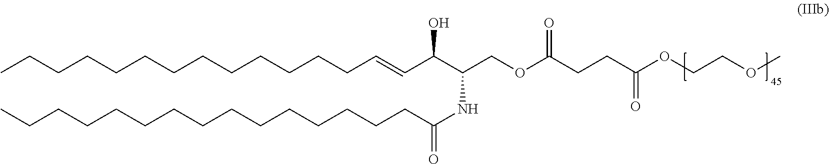

### EMBODIMENT 34

The composition of any one of embodiments 1 to 22, preferably any one of embodiments 19 to 22, wherein the PEGylated lipid is a PEGylated diacylglycerol of formula (IV)

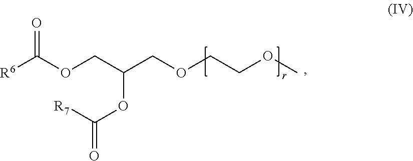

wherein each of R6 and R7 is individually and independently linear C11 -C17 alky!, and r is any integer from 18, 19 or 20, or from 44, 45 or 46 or from 113, 114 or 115.

### EMBODIMENT 35

The composition of embodiment 34, wherein R6 and R7 are the same.

### EMBODIMENT 36

The composition of embodiment 34, wherein R6 and R7 are different.

### EMBODIMENT 37

The composition of any one of embodiments 34 to 36, wherein each of R6 and R7 is individually and independently selected from the group consisting of linear C17 alkyl, linear C15 alkyl and linear C13 alkyl.

### EMBODIMENT 38

The composition of any one of embodiments 1 to 22 and 34 to 37, preferably one of embodiments 12 to 14, more preferably any one of embodiments 16 and 18, wherein the PEGylated diacylglycerol of formula (IV) is

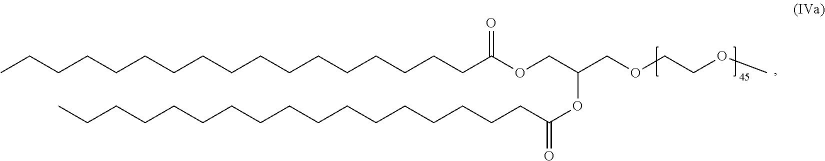

### EMBODIMENT 39

The composition of any one of embodiments 1 to 22 and 34 to 36, preferably one of embodiments 12 to 14, more preferably any one of embodiments 16 and 18, wherein the PEGylated diacylglycerol of formula (IV) is

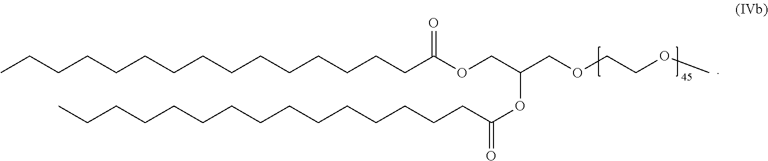

### EMBODIMENT 40

The composition of any one of embodiments 1 to 22 and 34 to 36, preferably one of embodiments 12 to 14, more preferably any one of embodiments 16 and 18, wherein the PEGylated diacylglycerol of formula (IV) is

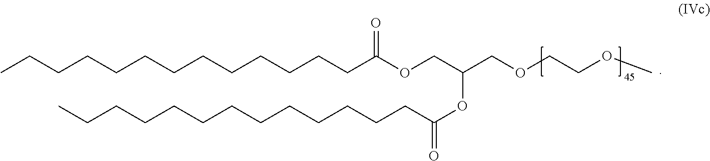

### EMBODIMENT 41

The composition of any one of embodiments 1 to 40, wherein the cationic lipid of formula (I) is selected from the group consisting of

β-arginyl-2,3-diamino propionic acid-N-palmityl-N-oleyl-amide trihydroehloride

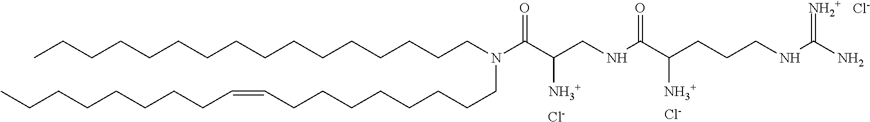 [SMILES: CCCCCCCC/C=C\CCCCCCCCN(CCCCCCCCCCCCCCCC)C(=O)C([NH3+])CNC(=O)C([NH3+])CCCNC(N)=[NH2+] ; [Cl-] ; [Cl-] ; [Cl-]]

β-arginyl-2,3-diamino propionic acid-N-lauryl-N-myristyl-amide trihydroehloride

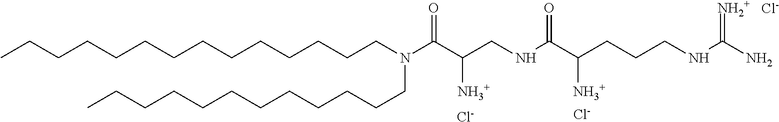 [SMILES: CCCCCCCCCCCCCCN(CCCCCCCCCCCC)C(=O)C([NH3+])CNC(=O)C([NH3+])CCCNC(N)=[NH2+] ; [Cl-] ; [Cl-] ; [Cl-]]

and
ϵ-arginyl-lysine-N-lauryl-N-myristyl-amide trihydrochloride

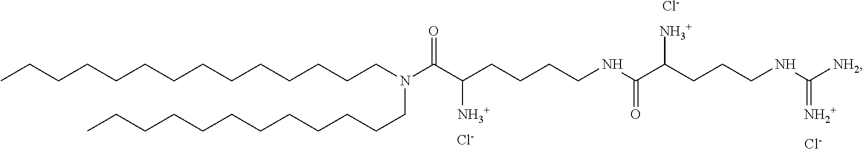 [SMILES: [Cl-] ; CCCCCCCCCCCCCCN(CCCCCCCCCCCC)C(=O)C([NH3+])CCCCNC(=O)C([NH3+])CCCNC(N)=[NH2+] ; [Cl-] ; [Cl-]]

wherein the sterol compound is selected from the group consisting of cholesterol and stigmasterin; and
wherein the PEGylated lipid is a PEGylated phosphoethanolamine of formula (II), wherein the PEGylated phosphoethanolamine is selected from the group consisting of
1,2-distearoyl-sn-glycero-3-phosphoethanolamine-N-[methoxy(polyethylene glycol)-2000] (ammonium salt)

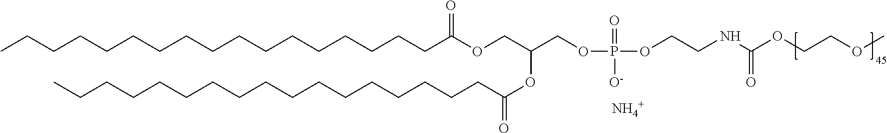

1,2-distearoyl-sn-glycero-3-phosphoethanolamine-N-[methoxy(polyethylene glycol)-5000] (ammonium salt)

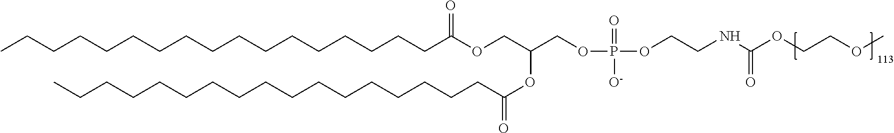

### EMBODIMENT 42

The composition of any one of embodiments 1 to 40, wherein the cationic lipid of formula (I) is selected from the group consisting of

β-arginyl-2,3diamino propionic acid-N-palmityl-N-oleyl-amide trihydrochloride

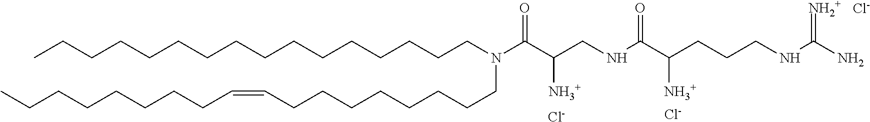 [SMILES: CCCCCCCC/C=C\CCCCCCCCN(CCCCCCCCCCCCCCCC)C(=O)C([NH3+])CNC(=O)C([NH3+])CCCNC(N)=[NH2+] ; [Cl-] ; [Cl-] ; [Cl-]]

β-arginyl-2,3-diamino propionic acid-N-lauryl-N-myristyl-amide trihydrochloride

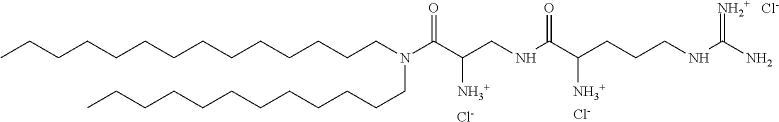 [SMILES: CCCCCCCCCCCCCCN(CCCCCCCCCCCC)C(=O)C([NH3+])CNC(=O)C([NH3+])CCCNC(N)=[NH2+] ; [Cl-] ; [Cl-] ; [Cl-]]

and
ϵ-arginyldysine-N-lauryl-N-myristyl-amide trihydrochloride

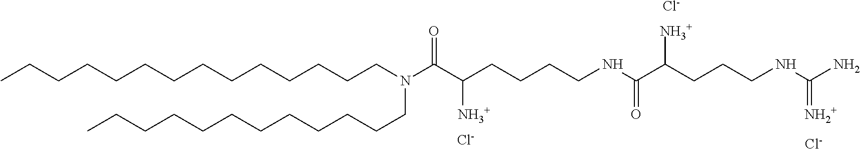 [SMILES: [Cl-] ; CCCCCCCCCCCCCCN(CCCCCCCCCCCC)C(=O)C([NH3+])CCCCNC(=O)C([NH3+])CCCNC(N)=[NH2+] ; [Cl-] ; [Cl-]]

wherein the sterol compound is selected from the group consisting of cholesterol and stigmasterin; and
wherein the PEGylated lipid is a PEGylated ceramide of formula (III), wherein the PEGylated ceramide is selected from the group consisting of
N-octanoyl-sphingosine-1 -{succinyl[methoxy(polyethylene glycol)2000]}

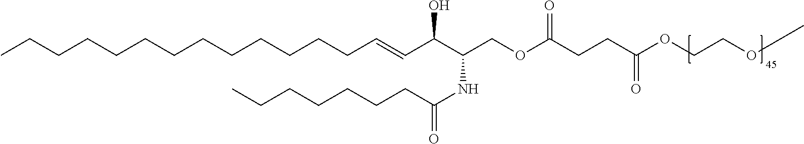

and
N-palmitoyl-sphingosine-1-{succinyl[methoxy(polyethylene glycol)2000]}

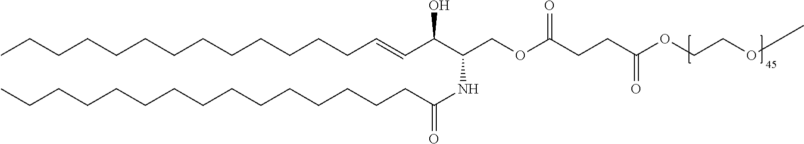

### EMBODIMENT 43

The composition of any one of embodiments 1 to 40, wherein

the cationic lipid of formula (I) is selected from the group consisting of
β-arginyl-2,3-diamino propionic acid-N-palmityl-N-oleyl-amide trihydrochloride

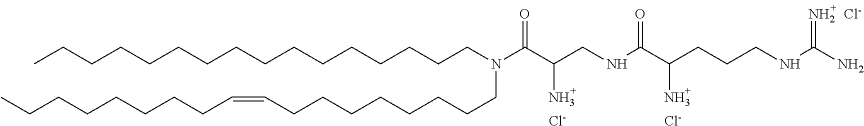 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

β-arginyl-2,3-diamino propionic acid-N-lauryl-N-myristyl-amide trihydrochloride

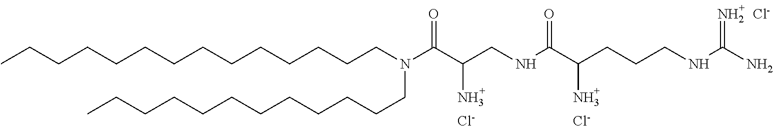 [SMILES: CCCCCCCCCCCCCCN(CCCCCCCCCCCC)C(=O)C(N)CNC(=O)C(N)CCCNC(=N)N ; [Cl-] ; [Cl-] ; [Cl-]]

and ϵ-arginyl-lysine-N-lauryl-N-myristyl-amide trihydrochloride

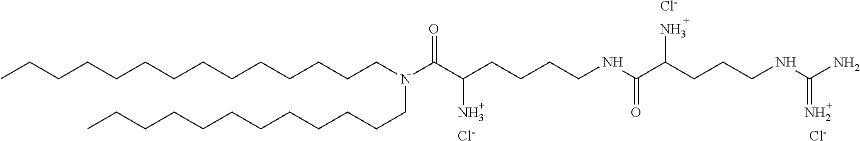 [SMILES: [Cl-] ; CCCCCCCCCCCCCCN(CCCCCCCCCCCC)C(=O)C(N)CCCCNC(=O)C(N)CCCNC(=N)N ; [Cl-] ; [Cl-]]

wherein the sterol compound is selected from the groups consisting of cholesterol and stigmasterin; and
wherein the PEGylated lipid is a PEGylated diacylglycerol of formula (IV), wherein the PEGylated diacylglycerol is selected from the group consisting of
1,2-Distearoyl-sn-glycerol [methoxy(polyethylene glycol)2000]

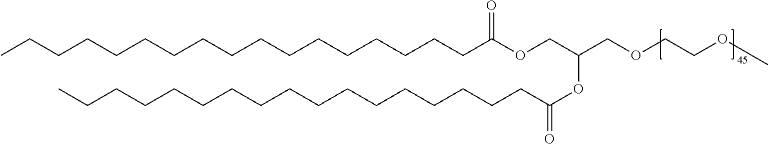

1,2-Dipalmitoyl-sn-glycerol [methoxy(polyethylene glycol)2000]

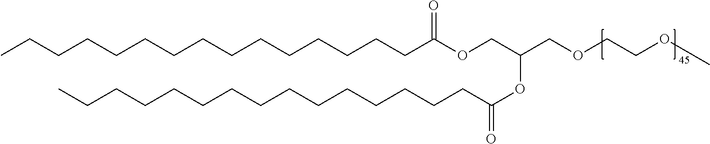

### EMBODIMENT 44

The composition of any one of embodiments 1 to 43, preferably of any one of embodiments 41 to 43, wherein

the cationic lipid is β-arginyl-2,3-diamino propionic acid-N-palmityl-N-oleyl-amide trihydrochloride

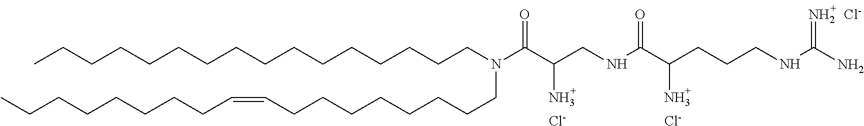 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

the sterol compound is cholesterol, and
the PEGylated lipid is PEGylated phosphoethanolamine of formula (II) is
1,2distearoyl-sn-glycero-3-phosphoethanolamine-N-[methoxy(polyethylene glycol)-2000] (ammonium salt)

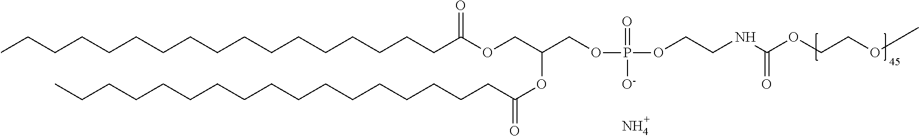

### EMBODIMENT 45

The composition of any of embodiments 1 to 44, preferably any of embodiments 41 to 44 and more preferably embodiment 44, wherein, in the lipid composition, the content of the cationic lipid composition is from about 65 mole % to about 75 mole %, the content of the sterol compound is from about 24 mole % to about 34 mole % and the content of the PEGylated lipid is from about 0.5 mole % to about 1.5 mole %, wherein the sum of the content of the cationic lipid, of the sterol compound and of the PEGylated lipid for the lipid composition is 100 mole %.

### EMBODIMENT 46

The composition of embodiment 45, wherein, in the lipid composition, the content of the cationic lipid is about 70 mole %, the content of the sterol compound is about 29 mole % and the content of the PEGylated lipid is about 1 mole %.

### EMBODIMENT 47

A composition of any of the preceding embodiments, wherein the lipid composition is as follows:

70 mole % of p-arginyl-2,3-diamine propionic acid-N-palmityl-N-oleyl-amide trihydrochloride

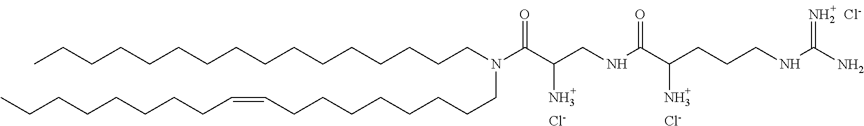 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

29 mole % of cholesterol, and
1 mole % of
1,2-distearoyl-sn-glycero-3-phosphoethanolamine-N-[methoxy(polyethylene glycol)-2000] (ammonium salt)

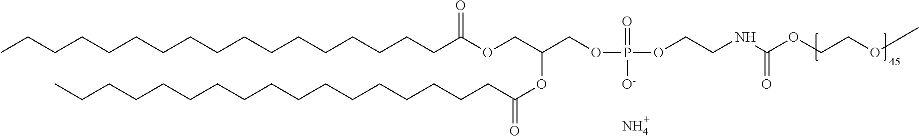

### EMBODIMENT 48

The composition of any one of embodiments 1 to 47, wherein the composition comprises a carrier, preferably the carrier is a pharmaceutically acceptable carrier.

### EMBODIMENT 49

The composition of embodiment 48, wherein the carrier is selected from the group comprising water, an aqueous solution, preferably an isotonic aqueous solution, a salt solution, preferably an isotonic salt solution, a buffer, preferably an isotonic buffer, and a water miscible solvent.

### EMBODIMENT 50

The composition, of embodiment 49, wherein the carrier is a water miscible solvent and wherein the water miscible solvent is selected from the group consisting of ethanol and tertiary butanol

### EMBODIMENT 51

The composition of any one of embodiments 48 to 50, wherein the carrier is an aqueous sucrose solution, preferably a 270 mM aqueous sucrose solution.

### EMBODIMENT 52

The composition according to any one of the preceding embodiments,

wherein the lipid composition is as follows:
70 mole % of P-arginyl-2,3-diamino propionic acid-N-palmityl-N-oleyl-amide trihydrochloride

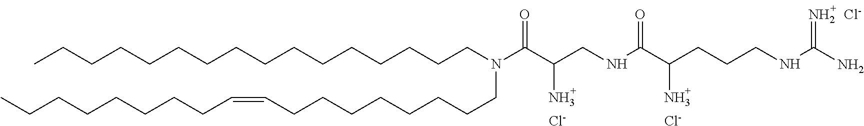 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

29 mole % of cholesterol, and
1 mole % of PEGylated phosphoethanolamine of formula (II) is;
1,2-distearoyl-sn-glycero-3-phosphoethanolamine-N-[methyl(polyethylene glycol)-2000] (ammonium salt)

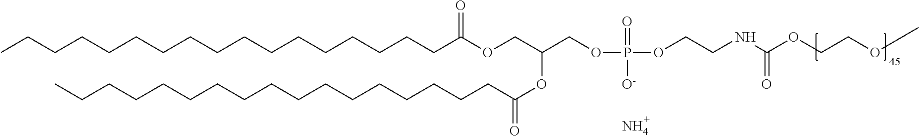

and
wherein the composition comprises a 270 mM aqueous sucrose solution.

### EMBODIMENT 53

The composition according to any one of embodiments 48 to 52, wherein the lipid composition forms particles in the carrier.

### EMBODIMENT 54

The composition of embodiment 53, wherein the particles have a Z-average size according to DLS measurement of about 30 nm to about 150 nm.

### EMBODIMENT 55

The composition of embodiment 54, wherein the particles have a Z-average size according to DLS measurement of about 50 nm to about 100 nm.

### EMBODIMENT 56

The composition of any one of embodiments 53 to 55, wherein the Z-average size according to DLS measurement of the particles is about 60-80 nm as determined by dynamic light scattering.

### EMBODIMENT 57

The composition of any one of embodiments 1 to 56, preferably any one of embodiments 48 to 56, wherein the composition, determined at a temperature of 20° C. and in a 270 mM sucrose solution, has a zeta potential of about +25 to about +80 mV, preferably of about +30 mV to about +60 mV, more preferably of about +46 mV.

### EMBODIMENT 58

The composition of any one of embodiments 1 to 57, preferably any one of embodiments 41 to 57 and more preferably any one of embodiments 47 and 52, wherein the composition farther comprises a chemical compound, wherein the chemical compound is a biologically active agent or a pharmaceutically active agent.

### EMBODIMENT 59

The composition of any one of embodiments 1 to 57, preferably any one of embodiments 41 to 57 and more preferably any one of embodiments 47 and 52, wherein the composition further comprises a chemical compound, wherein the chemical compound is capable of being delivered by the lipid composition into and/or to a cell.

### EMBODIMENT 60

The composition of embodiment 59, wherein the cell is a cell of a mammal, preferably the mammal is selected from the group comprising man, mouse, rat, rabbit, hamster, guinea pig, monkey, dog, cat, pig, sheep, goat, cow and horse.

### EMBODIMENT 61

The composition of any one of embodiments 59 to 60, wherein the cell is a pulmonary endothelial cell, preferably the cell is a human pulmonary endothelial cell.

### EMBODIMENT 62

The composition of any one of embodiments 58 to 61, wherein the chemical compound is selected from the group comprising an oligonucleotide, a polynucleotide, a nucleic acid, a peptide, a polypeptide, a protein and a small molecule.

### EMBODIMENT 63

The composition of embodiment 62, wherein the chemical compound is a nucleic acid and wherein the nucleic acid is selected from the group comprising RNA, DNA, PNA and LNA,

### EMBODIMENT 64

The composition of embodiment 62, wherein the nucleic acid is a functional nucleic, acid, preferably the functional nucleic acid is selected from the group comprising an siRNA, a microRNA, an siNA, a RNA interference mediating nucleic acid, an antisense nucleic acid, a ribozyme, an aptamer a spiegelmer and mRNA.

### EMBODIMENT 65

The composition of embodiment 62, wherein the polynucleotide is selected from the group comprising an siRNA, a microRNA, an siNA, a RNA interference mediating nucleic acid, an antisense nucleic acid, a ribozymes an aptamer, a spiegelmer and mRNA.

### EMBODIMENT 66

The composition of embodiment 62, wherein the oligonucleotide is selected from the group comprising an siRNA, a microRNA, an siNA, a RNA interference mediating nucleic acid, an antisense nucleic acid, a ribzyme, an aptamer and a spiegelmer.

### EMBODIMENT 67

The composition of any one of embodiments 62 and 66, wherein the oligonucleotide forms a complex with the lipid composition.

### EMBODIMENT 68

The composition of any one of embodiments 1 to 67, preferably of any one of embodiments 41 to 47 and 53 to 56, wherein the composition comprises an siRNA molecule.

### EMBODIMENT 69

The composition of embodiment 68, wherein the siRNA molecule is targeting ANG2.

### EMBODIMENT 71

Use composition of embodiment 70, wherein the ANG2 targeting siRNA molecule comprises one or both of the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides indicated as capital letter are 2-O-methyl,

### EMBODIMENT 71

The composition of embodiment 70, wherein the ANG 2 targeting siRNA molecule comprises the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides indicated as capital letter are 2′-O methyl.

### EMBODIMENT 72

The composition of embodiment 62, wherein the chemical compound is a protein and wherein the protein is selected from the group comprising an antibody, a cytokine and an anticaline.

### EMBODIMENT 73

The composition of any one of the preceding embodiments, wherein the lipid composition is as follows:

70 mole % of P-arginyl-2,3-diamino propionic acid-N-palmityl-N-oleyl-amide trihydrochloride

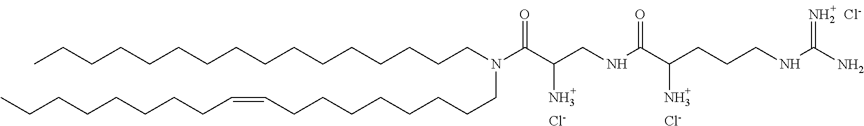 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

29 mole % of cholesterol, and
1 mole % of
1,2-distearoyl-sn-glycero-3-phosphoethanolamine-N-[methoxy(polyethylene glycol)-2000] (ammonium salt)

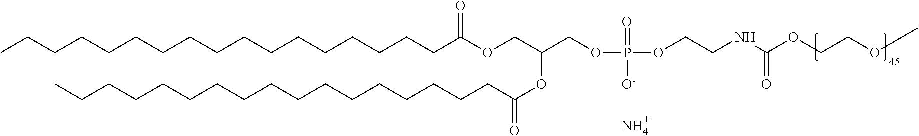

wherein the composition comprises, preferably as a or the carrier, a 270 mM aqueous sucrose solution; and
wherein the composition comprises a chemical compound, wherein the chemical compound is selected from the group comprising an siRNA, microRNA, siNA and a RNA interference mediating compound, preferably the chemical compound is (a) a or the biologically or pharmaceutical active agent and/or (b) is capable of being delivered by the lipid composition into and/or to a cell, more preferably to a mammalian pulmonary endothelial cell.

### EMBODIMENT 74

The composition according to any one of embodiments 1 to 73, preferably any one of embodiments 58 to 73 and more preferably embodiment 73, wherein the chemical compound is a junctional nucleic acid and wherein the ratio between the charged lipid nitrogen atoms to the nucleic acid backbone phosphates (N/P ratio) is about from 3 to 12, preferably about from 5 to 10 and more preferably about from 8 to 9 and most preferably about 8.4.

### EMBODIMENT 75

The composition of any one of embodiments 73 and 74, wherein the composition comprises an siRNA molecule.

### EMBODIMENT 76

The composition of embodiment 75, wherein the siRNA molecule is targeting ANG2.

### EMBODIMENT 77

The composition of embodiment 76, wherein the ANG2 targeting siRNA molecule comprises one or both of the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides indicated as capital letter are 2′-O-methyl.

### EMBODIMENT 78

The composition of embodiment 77, wherein the ANG 2 targeting siRNA molecule comprises the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides Indicated as capital letter are 2′-O-methyl.

### EMBODIMENT 79

The composition of any one of embodiments 73 to 78, preferably of embodiment 78, wherein the composition comprises 0.28 mg/ml of siRNA and 2.4 mg/ml total lipids.

### EMBODIMENT 80

The composition of any one of the preceding embodiments, wherein the lipid composition is as follows:

70 mole % of β-arginyl-2,3-diamino propionic acid-N-palmityl-N-oleyl-amide trihydrochloride

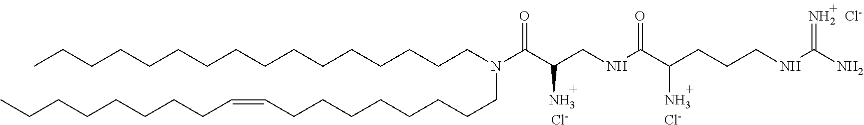

29 mole % of cholesterol, and
1 mole % of
1,2-distearoyl-sn-glycero-3-phosphoethanolamine-N-[methoxy(polyethylene glycol)-2000](ammonium salt)

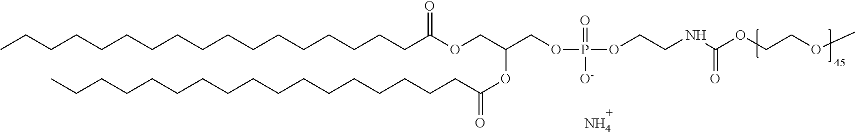

wherein the composition comprises, preferably as a or the carrier, a 270 mM aqueous sucrose solution;
wherein the composition comprises a chemical compound, wherein the chemical compound is selected -from the group comprising an siRNA, microRNA and siNA, preferably the chemical compound is (a) a or the biologically or pharmaceutically active agent and/or (b) is capable of being delivered by the lipid composition into and/or to a cell, more preferably to a mammalian pulmonary endothelial cell; and
wherein the ratio between the charged lipid nitrogen atoms to the nucleic acid backbone phosphates (N/F ratio) is about 8 to 9, preferably about 8.4.

### EMBODIMENT 81

The composition of embodiment 80, wherein the composition comprises an siRNA molecule.

### EMBODIMENT 82

The composition of embodiment 81, wherein the siRNA molecule is targeting ANG2.

### EMBODIMENT 83

The composition of embodiment 82, wherein the ANG2 targeting siRNA molecule comprises one or both of the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides indicated as capital letter are 2′-O-methyl.

### EMBODIMENT 84

The composition of embodiment 83, wherein the ANG 2 targeting siRNA molecule comprises the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

preferably the nucleotides indicated as capital letter are 2′-O-methyl.

### EMBODIMENT 85

The composition of any one of embodiments 80 to 84, preferably of embodiment 84, wherein the composition comprises 0.28 mg/ml of siRNA and 2.4 mg/ml total lipids.

### EMBODIMENT 86

The composition of any one of embodiments 1 to 85, preferably of any one of embodiments 58 to 85 and more preferably of any one of embodiments 73 to 85, for use in a method for the treatment and/or prevention of a disease of a subject.

### EMBODIMENT 87

The composition of embodiment 86, wherein the method comprises administering to a subject, in need thereof an effective amount of the composition, preferably a therapeutically effective amount of the composition.

### EMBODIMENT 88

The composition of any one of embodiments 86 to 87, wherein the composition delivers the chemical compound into a cell of the subject.

### EMBODIMENT 89

The composition of embodiment 88, wherein the cell is a pulmonary endothelial cell.

### EMBODIMENT 90

The composition of embodiment 89, wherein the chemical compound provides for a therapeutic effect in the pulmonary endothelial cell, preferably the chemical compound targets and more preferably inhibits a target molecule within the cell, whereupon the therapeutic effect is achieved.

### EMBODIMENT 91

The composition of embodiment 90, wherein the target molecule is involved in the pathological mechanism underlying the disease.

### EMBODIMENT 92

The composition of any one of embodiments 86 to 91, wherein the disease is selected from the group comprising acute lung injury, acute respiratory distress syndrome, lung cancer, pulmonary metastasis, pulmonary hypertension and pulmonary artery hypertension.

### EMBODIMENT 93

The composition of any one of embodiments 86 to 92, wherein the subject is selected from the group comprising man, mouse, rat, rabbit, hamster, guinea pig, monkey, dog, cat, pig, sheep, goat, cow and horse.

### EMBODIMENT 94

The composition of any one of embodiments 86 to 93, wherein the composition is administered to the subject by means of intravenous administration or by means of inhalation.

### EMBODIMENT 95

Use of the composition of any one: of embodiments 1 to 85, preferably of any one of embodiments 58 to 85 and more preferably of any one of embodiments 73 to 85, for the manufacture of a medicament for the treatment and/or prevention of a disease.

### EMBODIMENT 96

Use of embodiment 95, wherein the disease is a disease where a target molecule involved in the pathological mechanism underlying the disease is present in a pulmonary endothelial cell and the inhibition of the target molecule provides a therapeutic effect.

### EMBODIMENT 97

Use of any one of embodiments 95 to 96, wherein the chemical compound of the composition targets and more preferably inhibits the target molecule within the cell, whereupon the therapeutic effect is achieved,

### EMBODIMENT 98

Use of any one of embodiments 95 to 97, wherein the disease is selected from the group comprising acute lung injury, acute respiratory distress syndrome, lung cancer, pulmonary metastasis, pulmonary hypertension and pulmonary artery hypertension.

### EMBODIMENT 99

Use of any one of embodiments 95 to 98, wherein the medicament is for intravenous administration,

### EMBODIMENT 100

A pharmaceutical composition comprising a composition of any one of embodiments 1 to 85, preferably a composition of any one of embodiments 58 to 85 and more preferably of embodiments 73 to 85, and a pharmaceutically acceptable carrier.

### EMBODIMENT 101

The pharmaceutical composition of embodiment 100, wherein the chemical composition of the composition of any one of embodiments 1 to 85, preferably a composition of any one of embodiments 58 to 850 and more preferably of embodiments 73 and 85, is a or the pharmaceutically active agent.

### EMBODIMENT 102

The pharmaceutical composition of any one of embodiments 100 to 101, wherein the carrier of the composition of any one of embodiments 1 to 85, preferably a composition of any one of embodiments 57 to 85 and more preferably of embodiments 73 to 85, is a or the pharmaceutically acceptable carrier.

### EMBODIMENT 103

The pharmaceutical composition of any one of embodiments 100 to 102, wherein the pharmaceutical composition is for use in the treatment and/or prevention of a disease, whereby the disease is defined as in any of embodiments 95 to 99.

### EMBODIMENT 104

Use of the composition of any one of embodiments 1 to 85, preferably of any one of embodiments 58 to 85 and more preferably of any one of embodiments 73 to 85, as a transferring agent.

### EMBODIMENT 105

Use of embodiment 104, wherein the transferring agent transfers a biologically active or pharmaceutically active compound into a cell, preferably a mammalian cell and more preferably a human cell.

### EMBODIMENT 106

Use of embodiment 105, wherein the cell is a pulmonary endothelial cell, preferably a human pulmonary endothelial cell.

### EMBODIMENT 107

Use of any of embodiments 104 to 106, wherein the chemical compound of the chemical composition is the biologically active agent or the pharmaceutical active agent.

### EMBODIMENT 108

A kit comprising a composition of any one of embodiments 1 to 85, preferably of any one of embodiments 58 to 85 and more preferably of any one of embodiments 73 to 85, and instructions of use.

### EMBODIMENT 109

A method for transferring a biologically active compound or a pharmaceutically active compound into a cell or across a membrane of a cell, wherein the method comprises the step of contacting the cell or the membrane of a cell with a composition of any one of embodiments 1 to 85, preferably of any one of embodiments 58 to 85 and more preferably of any one of embodiments 73 to 85, and the biologically active compound or pharmaceutically active compound,

EMBODIMENT 110

The method of embodiment 109, wherein the method comprises the step of detecting the biologically active compound or the pharmaceutically active compound in the cell and/or beyond the membrane of a cell.

### EMBODIMENT 111

The method of any one of embodiments 109 to 110, wherein the biologically active compound or the pharmaceutically active compound is the chemical compound of the composition of any one of embodiments 58 to 85.

### EMBODIMENT 112

A method for the treatment and/or prevention of a disease, wherein the method comprises administering to a subject in need thereof an effective amount of a composition of any one of embodiments 1 to 85, preferably of any one of embodiments 58 to 85 and more preferably of any one of embodiments 73 to 85.

### EMBODIMENT 113

The method of embodiment 112, wherein the composition delivers the chemical compound into a cell of the subject.

### EMBODIMENT 114

The method of embodiment 113, wherein the cell is a pulmonary endothelial cell.

### EMBODIMENT 115

The method of embodiment 114, wherein the chemical compound provides for a therapeutic effect in the pulmonary endothelial cell, preferably the chemical compound targets said more preferably inhibits a target molecule within the cell, whereupon the therapeutic effect is achieved.

### EMBODIMENT 116

The method of embodiment 115, wherein the target molecule is involved in the pathological mechanism underlying the disease.

### EMBODIMENT 117

The method of any one of embodiments 112 to 116, wherein the disease is selected from the group comprising acute lung injury, acute respiratory distress syndrome, lung cancer, pulmonary metastasis, pulmonary hypertension, and pulmonary artery hypotension.

### EMBODIMENT 118

The method of any one of embodiments 112 to 117, wherein the subject is selected from the group comprising man, mouse, rat, rabbit, hamster, guinea pig, monkey, dog, cat, pig, sheep, goat, cow and horse, preferably the subject is man.

### EMBODIMENT 119

A method for the manufacture of a medicament, wherein the method comprises formulating a composition according to any one of embodiments 1 to 85 with a pharmaceutically active agent.

### EMBODIMENT 120

The method of embodiment 119, wherein the medicament is for the treatment and/or prevention of a disease as described in any of the preceding embodiments.

### EMBODIMENT 121

The method of any one of embodiments 119 to 120, wherein the pharmaceutically active agent is a compound suitable tor the treatment of a lung disease.

### EMBODIMENT 122

The composition according to any one of embodiments 1 to 68, wherein the siRNA is targeting a target as indicated in Table 1. In a preferred embodiment the target

is PKN3, whereby more preferably the siRNA comprises a nucleic acid strand having a nucleotide sequence of SEQ ID NO: 3 and/or nucleic acid strand having a nucleotide sequence of SEQ ID NO: 4, whereby the nucleotides of the nucleic acid strands are modified as indicated in Table 1, or are not or differently modified;
is CD31. whereby more preferably the siRNA comprises a nucleic acid strand having a nucleotide sequence of SEQ ID NO: 11 and/or nucleic acid strand having a nucleotide sequence of SEQ ID NO: 12, whereby the nucleotides of the nucleic acid strands are modified as indicated in Table 1, or arc not or differently modified;
is Tie2 whereby more preferably the siRNA comprises a nucleic acid strand having a nucleotide sequence of SEQ ID NO: 9 and/or nucleic acid strand having a nucleotide sequence of SEQ ID NO: 10, whereby the nucleotides of the nucleic acid strands are modified as indicated in Table 1, or are not or differently modified;
is KDR/VEGPR2, whereby more preferably the siRNA comprises a nucleic acid strand having a nucleotide sequence of SEQ ID NO: 13 and/or nucleic acid strand having a nucleotide sequence of SEQ ID NO: 14, whereby the nucleotides of the nucleic acid strands are modified as indicated in Table 15 or are not or differently modified;
is CDHS/VE-Cadherin, whereby more preferably the siRNA comprises a nucleic acid strand having a nucleotide sequence of SEQ ID NO: 15 and/or nucleic acid strand having a nucleotide sequence of SEQ ID NO: 16, whereby the nucleotides of the nucleic acid strands are modified as indicated in Table 1, or are not or differently modified; or
is BMPR2, whereby more preferably the siRNA comprises a nucleic acid strand having a nucleotide sequence of SEQ ID NO: 17 and/or nucleic acid strand having a nucleotide sequence of SEQ ID NO: 18, whereby the nucleotides of the nucleic acid strands are modified as indicated in Table 1, or are not or differently modified;

It is within the present invention that in each and any embodiment of each and any aspect of the invention where the cationic lipid is (3-arginyl-2,3-diamino propionic acid-N-palmityl-N-oleyl-amide, said β-arginyl-2,3-diamino propionic acid-N-palmityl-N-oleylamide is preferably β-arginyl-2,3-L-diamino propionic acid-N-palmityl-N-oleyl-amide.

It is within the present invention that in each and any embodiment of each and any aspect of the invention where the cationic lipid is β-arginyl-2,3diamino propionic acid-N-lauryl-N-myristyl-amide, said β-arginyl-2,3-diamino propionic acid-N-lauryl-N-myristyl-amide is preferably β-L-arginyl-2,3-L-diamino propionic acid-N-lauryl-N-myristyl-amide.

It is within the present invention that in each and any embodiment of each and any aspect of the invention where the cationic lipid is ϵ-arginyl-lysine-N-lauryl-N-myristyl-amide, said ϵ-arginyl-lysine-N-lauryl-N-myristyl-amide is preferably ϵ-L-arginyl-L-lysine-N-lauryl-N-myristyl-amide.

The present inventors have surprisingly found that a composition comprising a lipid composition, wherein the lipid composition consists of

a cationic lipid of formula (I)

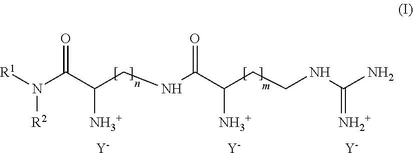

wherein n is any one of 1, 2, 3, and 4,
wherein m is any one of 1, 2 and 3,
wherein Y- is an anion, and
wherein each of R1 and R2 is individually and independently selected from the group consisting of linear C12-C18 alkyl and linear C12-C18 alkenyl;
   a sterol compound, wherein the sterol compound is selected from the group consisting of cholesterol and stigrnasterol; and
   a PEGylated lipid, wherein the PEGylated lipid comprises a PEG moiety and wherein the PEGylated lipid is selected from the group consisting of
a PEGylated phosphoetharsolamine of formula (II)

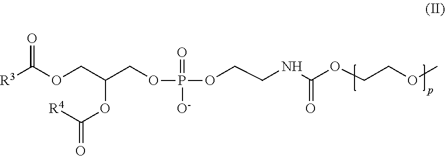

wherein each of R3 and R4 is individually and independently linear C13-C17 alkyl, and
wherein p is any integer from 15 to 130;
   a PEGylated cerarnide of formula (III)

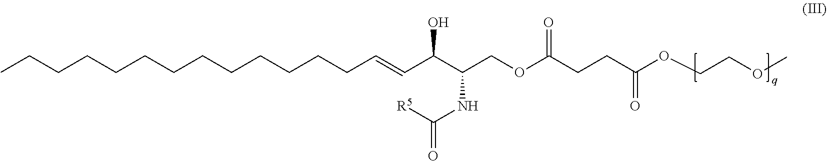

wherein R5 is linear C7-C15 alkyl, and
wherein q is any integer from 15 to 130; and
   a PEGylated diacylglycerol of formula (IV)

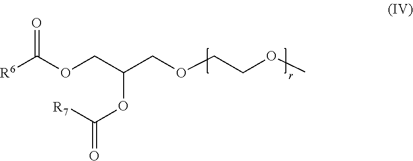

wherein each of R6 and R7 is individually and independently linear C11-C17 alkyl, and
wherein r is any integer from 15 to 130,
   is suitable to accumulate in and address lung and lung tissue of a host organism, such as a mammal, and thus to deliver an agent associated with such lipid composition such as a nucleic acid to such organ and tissue, and more specifically to pulmonary endothelial cells of such host organism. Preferably such nucleic acid is an oligonucleotide or a polynucleotide such as an siRNA, but is not limited thereto.

This finding is insofar surprising as it contrasts typical systemic behavior of cationic lipid nano-complexes, which generally show prevalent gene knockdown in liver tissue with only transient accumulation of siRNA in the lung (Polach, K J et al. (2012), Mol Ther 20: 91-100; Schroeder, A et al. (2010), J Intern Med 267: 9-21; Tao, W et al. (2010), Mol Ther 18: 1657-1666). Without wishing to be bound by any theory, the present inventors assume that the PEG moiety of the PEGylated lipid is present on the surface of the lipoplexes nanoparticles formed by the lipid composition forming a hydrophilic protective layer around the nanoparticles able to repel the adsorption of opsonin proteins via steric repulsion forces thus avoiding binding and rapid degradation of the lipoplexes by blood serum opsonins such as immunoglobulins and fibronectins which otherwise would result in systemic toxicity observed upon intravenous administration of cationic delivery systems. Furthermore, the cationic lipid was shown to allow for an active loading of negatively charged RNA into lipoplexes due to electrostatic interactions and eutropic effects, respectively. It condenses oligonucleotides, protects them from degradation and facilitates uptake by cells and endosomal release. The sterol compound of the lipid composition of the invention is assumed to maintain lipoplex stability without losing fusogenicity, and thus affects lamellarity, plasma pharmacokinetics and biodistribution of lipoplexes.

In light of the experimental evidence provided herein it is evident that the composition comprising a lipid composition of the invention is suitable for the treatment of lung diseases and disease which can be treated by targeting a pharmaceutically active agent and/or a therapeutically active agent to the lung, to lung issue and/or pulmonary endothelial cells. Such diseases include lung cancer as well as lung metastases (see, e.g. Steeg, P S (2006), Nat Med 12:895-904) whereby target molecules in case of lung cancer are CD31, Ras, myc, Hif-1a, VEGF-R2, -R1, R-3, PKN3, miR221, miR145. The observed low and one-time dosing observed with the composition comprising a lipid composition of the invention in particular also makes it a vehicle for the delivery of agents useful in the treatment of acute infections requiring hospital admission with intravenous drug administration, such as acute respiratory distress syndrome (ARDS)/acute lung injury (ALI), a life-threatening syndrome characterized by inflammation and increased vascular permeability leading to edema and sepsis (van der Heijden, M et al. (2009), Expert Opin Ther Targets 13:39-53; David, S et al. (2012), Crit Care Med 40: 3034-3041; and Hotchkiss, R S et al. (2003), N Engl J Med 348: 138-150. A particular target molecule which can be targeted by a pharmaceutically active agent or a therapeutically active agent in connection with ARDS/ALI, respectively, is ANG2. ANG2 which is also referred to as “angiopoietin-2” is an endothelial derived Tie2 antagonist and directly contributes to sepsis morbidity and mortality as a vascular destabilizing factor (Fiedler, U., and H. G. Augustin. 2006, Trends Immunol. 27:552-8). Selective target gene inhibition by the lipid, composition employing synthetic nucleic acids such as siRNAs, antisense molecules, antagomirs (described, for example, in Piva et al. 2013, INTERNATIONAL JOURNAL OF ONCOLOGY 43: 985-994, Ganguli et al, 2011, Bioinformation 7(1): 41-43 (2011), and Costa et al. 2013, Pharmaceuticals, 6, 1195-1220) and/or micro RNA mimics (described, for example, in Henry et al., 2011, Pharmaceutical research 12, 3030-3042; and Ling, H., et al. (2013), Nat Rev Drug Discov 12(11); 847-865) is therefore a strategy for the treatment and/or prevention of lung diseases, including acute life threatening lung diseases.

A further aspect of the present invention is a lipid composition, wherein the lipid composition consists of a cationic lipid of formula (I)

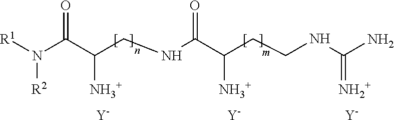

wherein n is any one of 1, 2, 3, and 4,
wherein m is any one of 1, 2 and 3,
wherein Y- is an anion, and
wherein each of R1 and R2 is individually and independently selected from the group consisting of linear C12-C18 alkyl and linear C12-C18 alkenyl;
   a sterol compound, wherein the sterol compound is selected from the group consisting of cholesterol and stigmasterol; and
   a PEGylated lipid, wherein, the PEGylated lipid comprises a PEG moiety and wherein the PEGylated lipid is selected from the group consisting of
   a PEGylated phosphoethanolamine of formula (II)

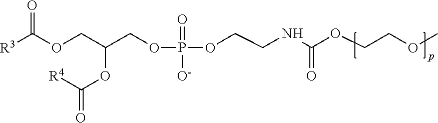

wherein each of R3 and R4 is individually and independently linear C13-C17 alkyl, and
wherein p is any integer from 15 to 130;
   a PEGylated ceramide of formula (III)

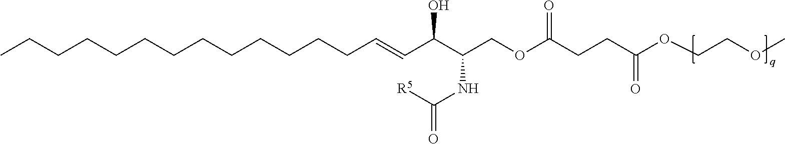

wherein RS is linear C7-C15 alkyl, and
wherein q is any integer from 15 to 130; and
a PEGylated diacylglycerol of formula (IV)

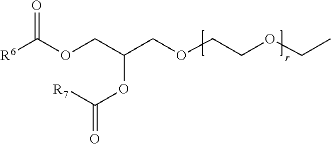

wherein each of R6 and R7 is individually and independently linear C11-C17 alkyl, and
wherein r is any integer from 15 to 130.

A further aspect of the invention resides in the use of the cationic lipid of formula (I)

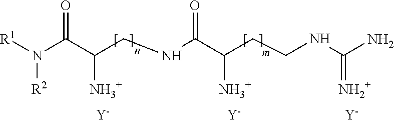

wherein n is any one of 1, 2, 3, and 4,
wherein m is any one of 1, 2 and 3,
wherein Y- is an anion, and
wherein each of R1 and R2 is individually and independently selected from the group consisting of linear C12-C18 alkyl and linear C12-C18 alkenyl,
   in the manufacture of a lipid composition of the invention, a medicament of the invention or a delivery vehicle of the invention.

A further aspect of the present invention is the use of a sterol compound selected from the group consisting of cholesterol and stigmasterol, in the manufacture of a lipid composition of the invention, a medicament of the invention or a delivery vehicle of the invention.

A still further aspect of the present invention is the use of a PEGylated phosphoethanolamine of formula (II)

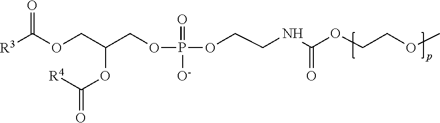

wherein each of R3 and R4 is individually and independently linear C13-C17 alkyl, and
wherein p is any integer from 15 to 130;
   a PEGylated ceramide of formula (III)

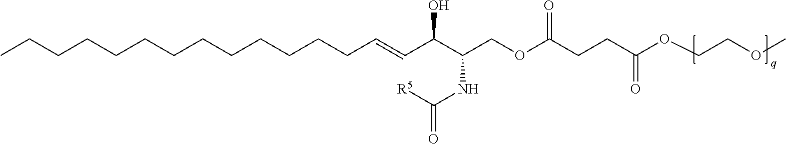

wherein R5 is linear C7-C15 alkyl, and
wherein q is any integer from 15 to 130; and
   a PEGylated diacylglycerol of formula (IV)

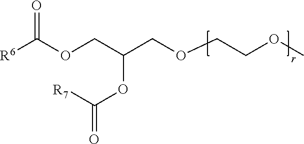

wherein each of R6 and R7 is individually and independently linear C11-C17 alkyl, and
wherein r is any integer from 15 to 130
   in the manufacture of a lipid composition of the invention, a medicament of the invention or a delivery vehicle of the invention.

If not indicated to the contrary, a composition of the invention is a composition comprising a lipid composition of the invention. It is within the present invention that any embodiment of the lipid composition of the invention is also an embodiment of the lipid composition of the invention.

As preferably used herein, a delivery agent or a delivery vehicle is a composition comprising the lipid composition of the invention. As also preferably used herein, a delivery agent or a delivery vehicle is a composition of the invention. A delivery agent or a delivery vehicle as preferably used herein is an agent or a vehicle such as a composition which is suitable to deliver a compound to a structure; preferably such structure is an organ, tissue or cell; more preferably such structure is an organ, tissue or cell. In a preferred embodiment such compound is a therapeutically active agent, a biologically active agent or a pharmaceutically active agent.

As preferably used herein, a therapeutically active agent is a compound which is suitable to elicit in a host organism a therapeutic or therapeutically beneficial effect.

As preferably used herein, a biologically active agent is a compound which is suitable to elicit in a host organism a biological effect.

As preferably used herein, a pharmaceutically active agent is a compound which is suitable to elicit in a host organism a pharmaceutical or pharmaceutically beneficial effect.

It will he acknowledged that if not indicated to the contrary, any embodiment of a therapeutically active agent is also an embodiment of a biologically active agent and of a pharmaceutically active agent, and vice versa.

In connection with the instant invention, therapy also encompasses prevention. In accordance therewith, a therapeutically active agent is, in an embodiment also an agent which is active in prevention of a disease. In an alternative embodiment, a therapeutically active agent is not active in the prevention of a disease.

As preferably used herein alkyl is an alkane substiuent missing one hydrogen, whereby an alkane consist only of hydrogen and carbon atoms, all bonds are single bonds, and the carbon atoms are not joined hi cyclic structures bat instead form an open chain; the general chemical formula of alkanes is CnH2n+2.

As preferably used herein alkenyl is an alkene substituent missing one hydrogen, whereby an alkene is an unsaturated chemical compound containing at least one carbon-carbon double bond.

As preferably used herein, PEG is polyethylene glycol.

As used herein, n is any integer between 1 and 4, which means that n may be 1, 2, 3 and 4. As used herein, m is any integer between 1 and 3, which means that m may be 1, 2 and 3.

The cationic lipid of the composition of the invention and a method for its preparation is, for example, disclosed in international patent application WO 2005/105152.

It is to be understood that the cationic lipid of the composition of the invention is a cationic lipids. More preferably, any of the NH or NH2 groups present in said lipid are present in a protonated form. Typically, any positive charge of said lipid is compensated by the presence of an anion. Such anion can be a monovalent or polyvalent anion. Preferred anions are halides, acetate and trifluoroacetate. Halides as used herein are preferably chlorides, fluorides, iodides and bromides. Most preferred are chlorides. Upon association of the cationic lipid and the therapeutically or pharmaceutically or biologically active compound to be transferred into a cell, the halide anion is replaced by the said active compound which preferably exhibits one or several negative charges, although it has to be acknowledged that the overall charge of the biologically active compound is not necessarily negative. The same considerations are equally applicable to the other compounds of the composition of the invention. In case such compound is as, anionic compound or bears one or several negative charges such negative charges may be compensated by the presence of a cation. Such cation can be a monovalent or polyvalent anion. Preferred cations are ammonium, sodium or potassium.

It is to be acknowledged that any compound according to formula (I) comprises at least two asymmetric C atoms. It is within the present invention that any possible diastereomer of such compound is disclosed herein, i.e. in particular the R—R; S—S; R—S and S—R diastereomer.

The sterol compound of the composition of the invention can be either synthetic or he obtained from natural sources such as sheep wool or plants.

The PEGylated lipid of the composition of the invention is available from commercial sources such as NOF Corporation, Japan; Avanti Polar Lipids, US; or Cordon Pharma, Switzerland.

Methods for determining the Z-average size of the lipid composition of the invention and the composition of the invention are known to the person skilled in the art and include Dynamic Light Scattering, DLS, as described in the example part or may be taken, for example, from the Zetasizer Nano Series User Manuel, Malvern Instruments Ltd., UK.

Methods for determining the zeta potential of the lipid composition of the invention and the composition of the invention are known to the person skilled in the art and include Electrophoretic Light Scattering which is described in the example part or may be taken, for example, from the Zetasizer Nano Series User Manuel, Malvern Instruments Ltd., UK.

The composition of the invention and particularly the lipid composition of the invention may comprise, in an embodiment, a carrier. Such carrier is preferably a liquid carrier. Preferred liquid carriers are aqueous carriers and non-aqueous carriers. Preferred aqueous carriers are water, an aqueous salt solution, an aqueous buffer system, more preferably the buffer system and/or the aqueous salt solution have a physiological buffer strength and physiological salt concentration(s). Preferred non-aqueous carriers are solvents, preferably organic solvents such as ethanol, tert-butanol. Without wishing to be bound by any theory, any water miscible organic solvent can, in principle, be used. It is to be acknowledged that the composition, more particularly the lipid composition can thus be present as or form liposomes; when contacted with overall negatively charged compounds, preferably compounds to be delivered by the lipid composition of the invention and/or the composition of the invention, the lipid composition of the invention and the composition of the invention form lipoplexes, i.e. a complex that is formed by the electrostatic interaction and the entropic effect based on the release of counter ions and water when a polyanion such as a nucleic acid molecule interact with a cationic lipid or a lipid system that contains beside other lipid components at least one cationic lipid component.

In a further embodiment, the lipid composition of the invention and/or the composition of the invention is present as a lyophilixed composition. The thus lyophilixed composition allows effective long-term storage of the composition at room temperature.

It is within the present invention that the lipid composition of the invention and/or the composition of the invention comprise a chemical compound, whereby said chemical compound is a biologically active agent, a therapeutically active agent and/or a pharmaceutically active agent. This kind of agent will also be referred as “the active agent” herein.

Preferably, any such active agent is a negatively charged molecule. The term negatively charged molecule means to include molecules that have at least one or more than one negatively charged group that can ion-pair with the positively charged group of the cationic lipid according to the present invention, although the present inventor does not wish to be bound by any theory. In principle, the positive charge at the linker moiety could also have some effect on the overall structure of either the lipid as such or any complex formed between the cationic lipid and the negatively charged molecule, i.e. the biologically active compound. Apart from that, the additional positive charge introduced into the lipid according to the present invention compared to the cationic lipids disclosed in U.S. Pat. No. 6,395,713, should contribute to an increased toxicity of this lipid as taught by Xu Y, Szoka F C Jr.; Biochemistry; May 7, 1996, 35 (18): 5616-23. In contrast to what the one skilled in the art would have expected from this document of the prior art the compounds according to the present invention are particularly suitable for the various purposes disclosed herein and are in particular devoid of any increased toxicity.

A peptide as preferably used herein is any polymer consisting of at least two amino acids which are covalently linked to each other, preferably through a peptide bond. More preferably, a peptide consists of two to ten amino acids. A particularly preferred embodiment of the peptide is an oligopeptide which even more preferably comprises from about 10 to about 100 amino acids. Proteins as preferably used herein are polymers consisting of a plurality of amino acids which are covalently linked to each other. Preferably such proteins comprise about at least 100 amino acids or amino acid residues.

A preferred protein which may be used in connection with the cationic lipid and the composition according to the present invention, is any antibody, preferably any monoclonal antibody.

Particularly preferred biologically active compounds, i.e. pharmaceutically active compounds and such further constituent as used in connection with the composition according to the present invention are nucleic acids. Such nucleic acids can be either DNA, RNA, PNA or any mixture thereof. More preferably, the nucleic acid is a functional nucleic acid. A functional nucleic acid as preferably used herein is a nucleic acid which is not a nucleic acid coding for a peptide and protein, respectively. Preferred functional nucleic acids are siRNA, siNA, RNAi, antisense-nucleic acids, ribozymes, aptamers and spiegelmers which are all known in the art.

siRNA are small interfering RNA as, for example, described in international patent application PCT/EP03/08666. These molecules typically consist of a double-stranded RNA structure which comprises between 15 to 25, preferably 18 to 23 nucleotide pairs which are base-pairing to each other, i.e. are essentially complementary to each other, typically mediated by Watson-Crick base-pairing. One strand of this double-stranded RNA molecule is essentially complementary to a target nucleic acid, preferably a mRNA, whereas the second strand of said double-stranded RNA molecule is essentially identical to a stretch of said target nucleic acid. The siRNA molecule may be flanked on each side and each stretch, respectively, by a number of additional oligonucleotides which, however, do not necessarily have to base-pair to each other.

RNAi has essentially the same design as siRNA, however,, the molecules are significantly longer compared to siRNA. RNAi molecules typically comprise 50 or more nucleotides and base pairs, respectively.

A further class of functional nucleic acids which are active based on the same mode of action as siRNA and RNAi is siNA. siNA is, e.g., described in international patent application PCT/BP03/074654. More particularly, siNA corresponds to siRNA, whereby the siNA molecule does not comprise any ribonucleotides.

Antisense nucleic acids, as preferably used herein, are oligonucleotides which hybridise based on base complementarity with a target RNA, preferably mRNA, thereby activating RNaseH. RNaseH is activated by both phosphodiester and phosphothioate-coupled DNA. Phosphodiester-coupled DNA, however, is rapidly degraded by cellular nucleases with exception of phosphothioate-coupled DNA. Antisense polynucleotides are thus effective only as DNA-RNA hybrid complexes. Preferred lengths of antisense nucleic acids range from 16 to 23 nucleotides. Examples for this kind of antisense oligonucleotides are described, among others, in U.S. Pat. No. 5,849,902 and U.S. Pat. No. 5,989,912.

A further group of functional nucleic acids are ribozymes which are catalytically active nucleic acids preferably consisting of RNA which basically comprise two moieties. The first moiety shows a catalytic activity, whereas the second moiety is responsible for the specific interaction with the target nucleic acid. Upon interaction between the target nucleic acid and the said moiety of the ribozyme, typically by hybridisation and Watson-Crick base-pairing of essentially complementary stretches of bases on the two hybridising strands, the catalytically active moiety may become active which means that it cleaves, either intramolecularly or intermolecularly, the target nucleic acid in case the catalytic activity of the ribozyme is a phosphodiesterase activity. Ribozymes, the use and design principles are known to the ones skilled in the art and, for example, described in Doherty and Doudna (Annu. Ref. Biophys. Biomolstruct, 2000; 30:457-75).

A still further group of functional nucleic acids are microRNAs, microRNA is a small non-coding RNA molecule. The completely processed mature miRNAs typically have a length of about 22 nucleotides. A mircoRNA functions in transcriptional and post-transcriptional regulation of gene expression. Encoded by eukaryotic nuclear DNA, miRNAs function via base-pairing with complementary sequences within mRNA molecules, usually resulting in gene silencing via translational repression or target degradation., and/or micro RNA mimics (see, for example, Anand, S. (2013), Vase Cell 5(1): 2; Kasinski, A. L. and F. J, Slack (2011), Nat Rev Cancer 11(12): 849-864; Liu, D. et al. (2011), Int J Dev Biol 55(4-5): 419-429; Staszel, T., et al. (2011), Pol Arch Med Wewn 121(10): 361-366; Urbich, C., et al. (2008), Cardiovasc Res 79(4): 581-588; Costa, P. M. and M. C, Pedroso de Lima (2013), Pharmaceuticals (Basel) 6(10): 1195-1220; and Henry, J. C., et al. (2011), Pharm Res 28(12): 3030-3042).

Another group of functional nucleic acids are antagonists which are, for example described in Costa, P. M. and M. C. Pedroso de Lima (2013), Pharmaceuticals (Basel) 6(10): 1195-1220; Piva, R., et al. (2013), Int J Oncol 43(4); 985-994; Ganguli, S., et al. (2011), Bioinformation 7(1): 41-43; and Ling, H., et al. (2013), Nat Rev Drug Discov 12(11): 847-865.

A still further group of junctional nucleic acids are aptamers. Aptamers are D-nucleic acids which are either single-stranded or double-stranded and which specifically interact with a target molecule. The manufacture or selection of aptamers is, e.g., described in European patent. EP 0 533 838. In contrast to RNAi, siRNA, siNA, antisense-nueleotides and ribozymes, aptamers do not degrade any target mRNA but interact specifically with the secondary and tertiary structure of a target compound such as a protein. Upon interaction with the target, the target, typically shows a change in its biological activity. The length, of aptamers typically ranges from as little as 15 to as much as 80 nucleotides, and preferably ranges from about 20 to about 50 nucleotides.

Another group of functional nucleic acids are spiegelmers as, for example, described in International patent application WO 98/08856. Spiegelmers are molecules similar to aptamers. However, spiegelmers consist either completely or mostly of L-nucleotides rather than D-nucleotides in contrast to aptamers. Otherwise, particularly with regard to possible lengths of spiegelmers, the same applies to spiegelmers as outlined in connection with aptamers.

A further aspect of the present invention is related to a pharmaceutical composition comprising the lipid composition of the invention or the composition of the invention. The pharmaceutical composition of the invention comprises a pharmaceutically active compound and optionally a pharmaceutically acceptable carrier. Such pharmaceutically acceptable carrier may, preferably, be selected from the group of carrier as defined herein in connection with the composition according to the present invention. It will be understood by those skilled In the art that any composition as described herein may, in principle, be also used as a pharmaceutical composition provided that its ingredients and any combination thereof is pharmaceutically acceptable. A pharmaceutical composition comprises a pharmaceutically active compound. Such pharmaceutically active compound can be the same as the further compound or the active agent of the composition according to the present invention which is preferably any biologically active compound, more preferably any biologically active compound as disclosed herein. The further constituent, pharmaceutically active compound and/or biologically active compound are preferably selected from the group comprising peptides, proteins, oligonucleotides, polynucleotides and nucleic acids.

The composition, particularly the pharmaceutical composition according to the present invention can be used for various forms of administration, whereby local administration and systemic administration are particularly preferred. Even more preferred is a route of administration which is selected from the group comprising intramuscular, percutaneous, subcutaneous, intravenous and pulmonary administration. As preferably used herein, local administration means that the respective composition is administered in close spatial relationship to the cell, tissue and organ, respectively, to which the composition and the biologically active compound, respectively, is to be administered. As used herein, systemic administration means an administration which is different from a local administration and more preferably is the administration into a body fluid such as blood and liquor, respectively, whereby the body liquid transports the composition to the cell, tissue and organ, respectively, to which the composition and the biologically active compound, respectively, is to be delivered.

As preferably used herein a host organism or a subject is a mammal, preferably the mammal is selected from the group comprising man, mouse, rat, rabbit, hamster, guinea pig, monkey, slog, cat, pig, sheep, goat, cow and horse; more preferably the host organism or the subject is man.

Any medicament: which can be manufactured using the composition according to the present invention, respectively, is for the treatment and prevention of a subject. Preferably such subject: is a mammal and even more preferably such mammal is selected from the group comprising man, mouse, rat, rabbit, hamster, guinea prig, monkey, dog, cat, pig, sheep, goat, cow and horse. In a further aspect the composition according to the present invention and/or the lipid composition of the invention can be used as a transferring agent, more preferably as a transfection agent.

As preferably used herein a transferring agent is any agent which is suitable to transfer a compound, more preferably a biologically active compound such as a pharmaceutically active compound across a membrane, preferably a cell membrane and more preferably transfer such compound into a cell as previously described herein. Preferably, the cells are pulmonary endothelial cells, more preferably endothelial cells of a host organism as defined herein.

In a still further aspect the present invention is related to a method for transferring, more particularly transfecting, a cell with a biologically active compound. In a first step, whereby the sequence of steps is not necessarily limited, the cell and the membrane and cell, respectively, is provided. In a second step, a compound according to the present invention is provided as well as a biologically active compound such as a pharmaceutically active compound. This reaction can be contacted with the cell and the membrane, respectively, and due to the biophysical characteristics of the compound and the composition according to the present invention, the biologically active compound will be transferred from one side of the membrane to the other one, or in case the membrane forms a cell, from outside the cell to within the cell. It is within the present invention that prior to contacting the cell and the membrane, respectively, the biologically active compound and composition of the invention and/or the lipid composition of the invention are contacted, whereupon preferably a complex is formed and such complex is contacted with the cell and the membrane, respectively.

In a further aspect of the present invention the method for transferring a biologically active compound and a pharmaceutically active compound, respectively, comprises the steps of providing the cell and the membrane, respectively, providing a composition according to the present invention and/or a lipid composition of the invention and contacting both the composition and the cell and the membrane, respectively. It is within the present invention that the composition may be formed prior or during the contacting with the cell and the membrane, respectively.

In an embodiment of any method for transferring a biologically active compound as disclosed herein, the method may comprise further steps, preferably the step of detecting whether the biologically active compound has beers transferred. Such detection reaction strongly depends on the kind of biologically active compounds transferred according to the method and will be readily obvious for the ones skilled in the art. It is within the present invention that such method is performed on any cell, tissue, organ and organism as described herein.

In a further aspect the present invention is related to a method for the manufacture of a medicament. The method comprises formulating a therapeutically active agent or a pharmaceutically active agent together with a composition of the invention and/or a lipid composition of the invention. Details on how such formulating is practiced are known to a person skilled in the art, and may also be taken from the example part of the instant description.

The instant invention is further illustrated by the following Figs, and Examples from which further features, embodiments and advantages of the invention maybe taken, whereby

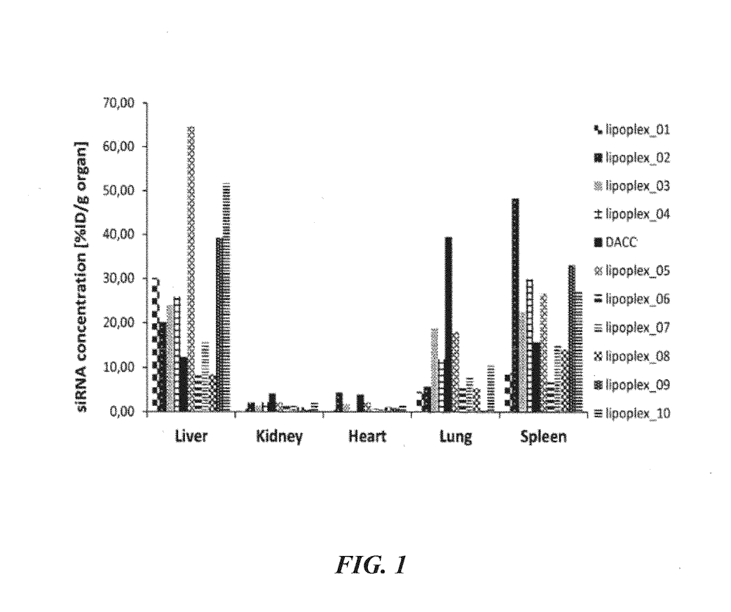

FIG. 1 is a diagram showing siRNA distribution in vivo 1 hour after systemic application using different lipoplex formulations; siRNA concentrations are indicated in percent initial dose per gram of tissue [% ID/g];

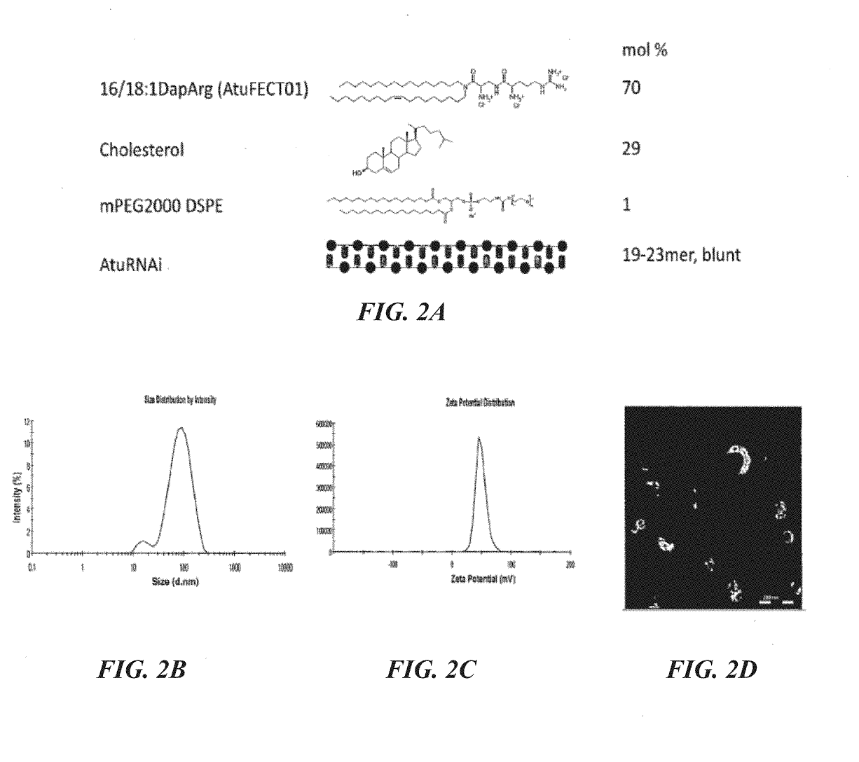

FIG. 2A is showing the chemical compounds forming DACC, their chemical structure and their molar ratio, expressed in percent, as well as the basic design of the siRNA molecules of the lipoplexes formed by DACC) and said siRNA moelcules; the siRNA molecules are blunt-ended and the circles on both strands indicate a 2-O-methyl modified nucleotide;

FIG. 2B is a diagram showing the particle size distribution of DACC/siRNA lipoplexes as Z-average size;

FIG. 2C is a diagram showing zeta potential of DACC/siRNA lipoplexes;

FIG. 2D is an electron microphotograph of DACC/siRNA lipoplexes;

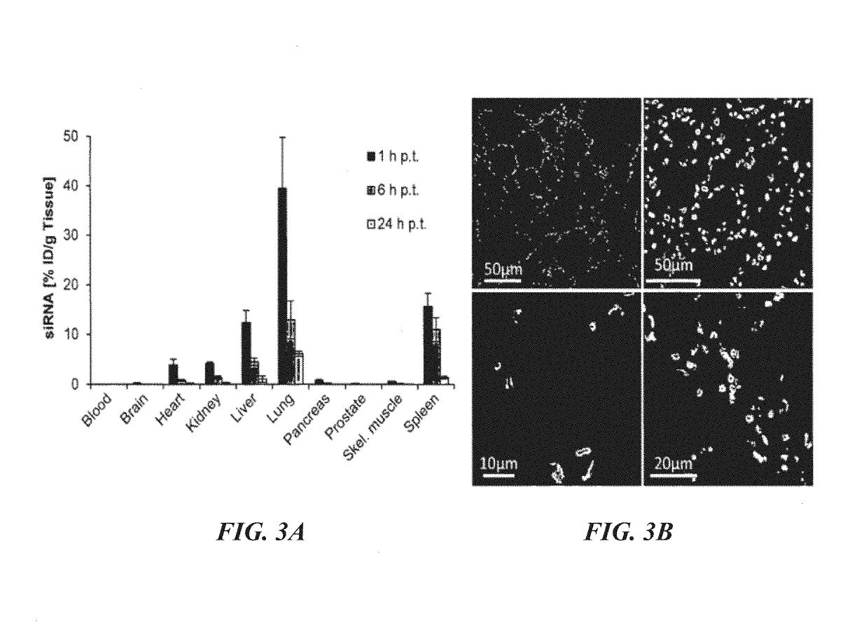

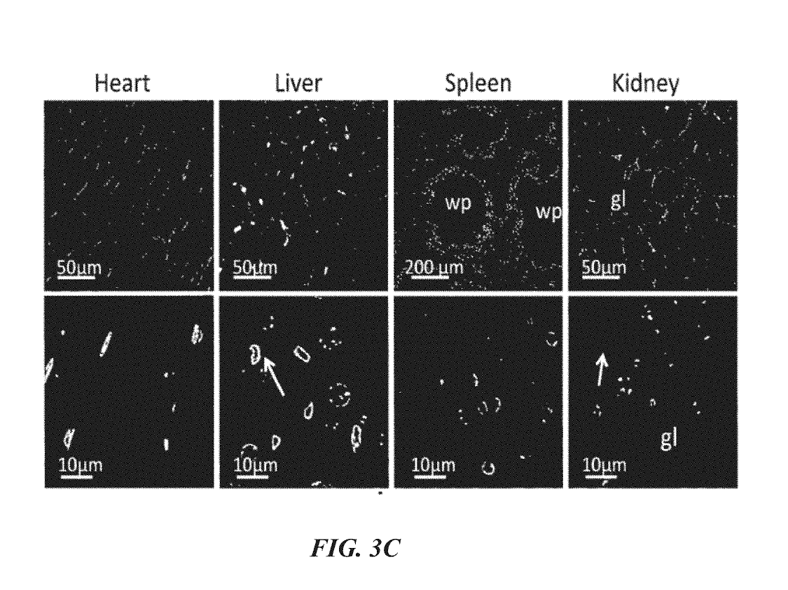

FIG. 3A is a diagram showing siRNA concentrations expressed as % ID/g tissue in various organs 1, 6 and 24 hours upon administration of DACC/siRNA lipoplexes;

FIG. 3B is a series of confocal microscopic images of formaline fixed paraffin embedded lung tissue sections illustrating cellular distribution of Cy3labeled siRNA in the lungs 1 hour alter systemic i.v. administration of DACC/siRNACy3; the upper left image shows siRNA-Cy3 staining in white, the upper right image and the lower images show close up views of siRNACy3 staining in red and nuclear staining in green; scale bars are indicated;

FIG. 3C is a series of confocal microscopic images of formaline fixed paraffin embedded sections of the heart, liver, spleen and kidney illustrating cellular distribution of Cy3-labeled siRNA in said organs 1 hour alter systemic i.v, administration of DACC/siRNACy3; the upper panel is depicted in white at low magnification, and the lower panel is in red in close up views; scale bars are indicated;

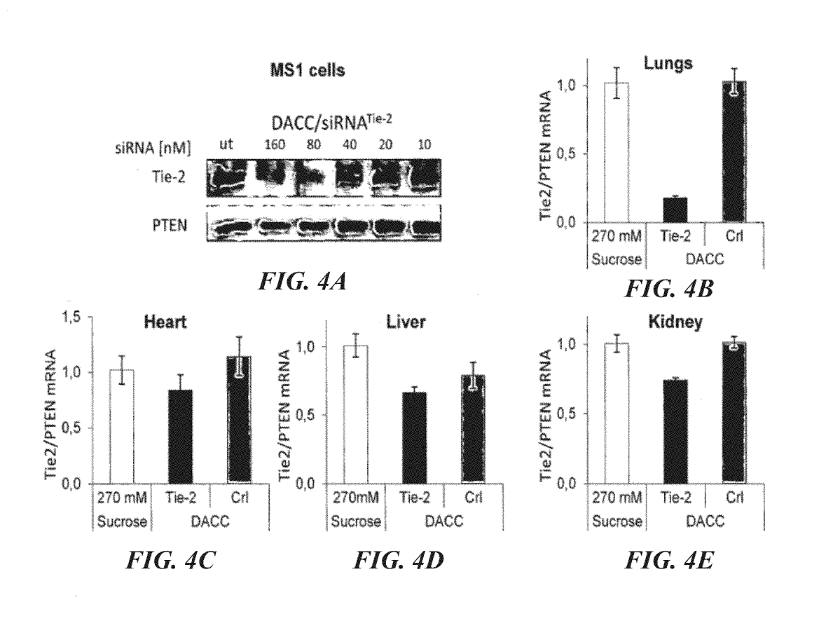

FIG. 4A shows the result of a Western blot analysis of a mouse endothelial cell line, MS-1, after transfection with DACC/siRNA^{Tie-2} lipoplexes for the inhibition of Tie-2;

FIGS. 4B to 4E are diagrams showing the inhibition of Tie-2 siRNA relative to the inhibition of PTEN mRNA in cells of the lung (FIG. 4B), beast (FIG. 4C), liver (FIG. 4D) and kidney (FIG. 4E) of mice upon treatment with DACC/siRNA^{Tie-2} lipoplexes as determined by quantitative reverse transcriptase-PCR analysis;

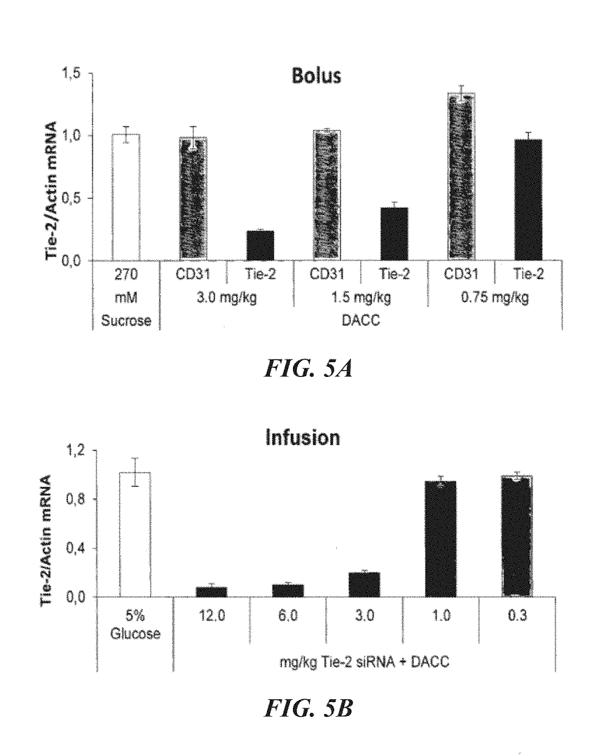

FIG. 5A is a diagram showing the inhibition of Tie-2 mRNA relative to the inhibition of Actin mRNA in lung cells of mice treated by bolus injection with DACC/siRNA^{Tie-2} lipoplexes, with DACC/siRNA^{CD31} lipoplexes and 270 mM sacrose solution, whereby the amount of lipoplexes administered was 3.0 mg/kg, 1.5 mg/kg and 0.75 mg/kg, respectively;

FIG. 5B is a diagram showing the inhibition of Tie-2 mRNA relative to the inhibition of Actin mRNA in lung cells of mice treated by infusion of DACC/siRNA^{Tie-2} lipoplexes and 5% glucose solution, whereby the amount of lipoplexes administered was 0.3 mg/kg, 1.0 mg/kg, 3.0 mg/kg, 6.0 mg/kg and 12 mg/kg, respectively;

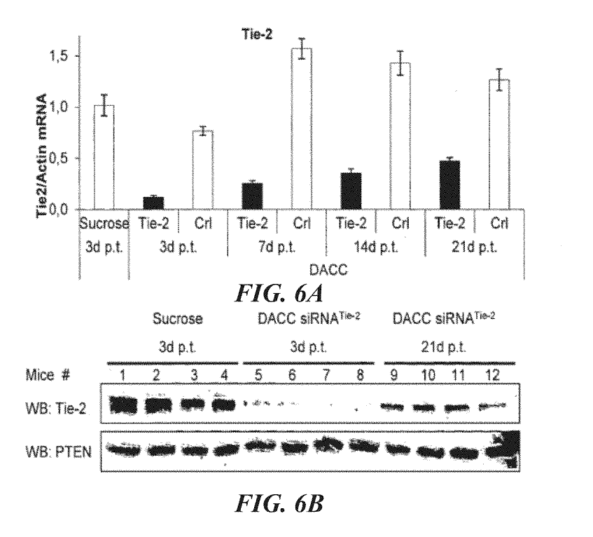

FIG. 6A is a diagram showing the inhibition of Tie-2 mRNA relative to the inhibition of Actin mRNA in lung cells of mice treated with DACC/siRNA^{Tie-2} lipoplexes, control lipoplexes or sucrose 3 days p.t., 7 days p.t., 14 days p.t. and 21 days p.t.;

FIG. 6B shows the result of a Western blot analysis of lung tissue of mice that was collected 3 days and 21 days after administration of a single dose of DACC/siRNA^{Tie-2} lipoplexes or sucrose solution, with PTEN being used as a loading control;

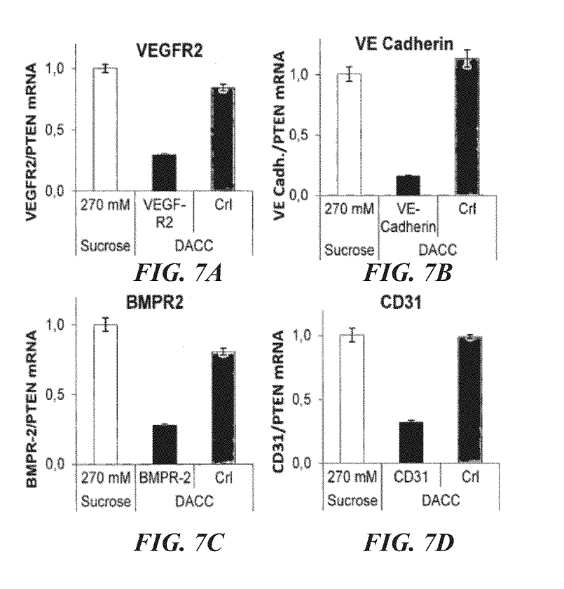

FIGS. 7A to 7D are diagrams showing the inhibition of various target genes (FIG. 7A; VEGFR2; FIG. 7B; VF-Cadherin; FIG. 7C: BMFR2; and FIG. 7D: CD31) relative to the inhibition of PTEN mRNA in pulmonary endothelium of mice treated with DACC/siRNA^{VEGFR2} lipoplexes (FIG. 7A), DACC/siRNA^{VE-Cadherin} lipoplexes (FIG. 7B), DACC/siRNA^{BMPR2} lipoplexes (FIG. 7C) and DACC/siRNA^{CD31} lipoplexes (FIG. 7D), whereby in each case 270 mM sucrose and a DACC/siRNA was administered in a control group of animals;

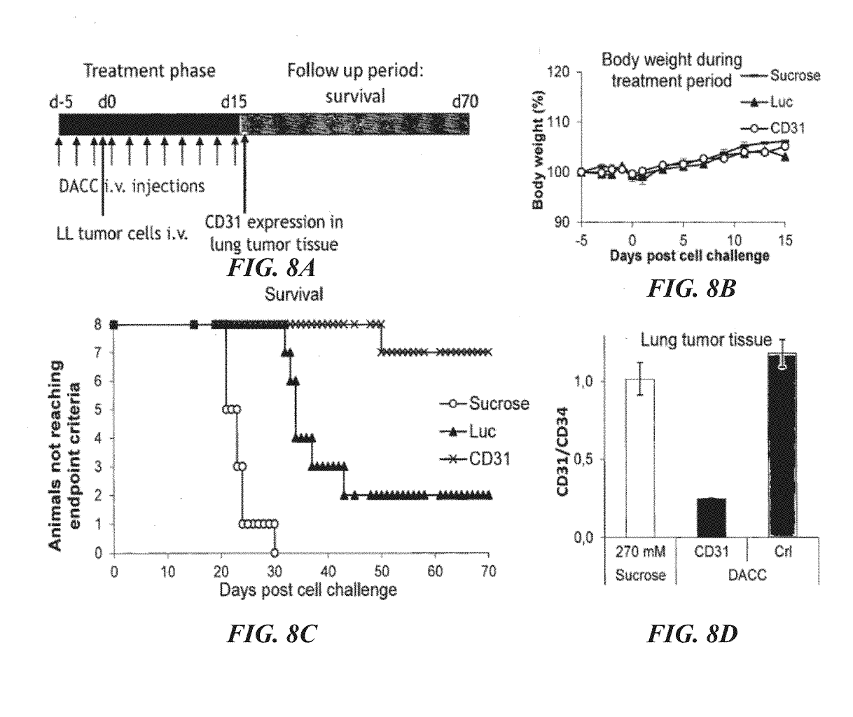

FIG. 8A is a representation of an experimental set-up for a lung metastasis mouse model and a treatment scheme using DACC/siRNA^{CD31};

FIG. 8B is a diagram illustrating relative body weight until day 15 during the treatment scheme shown in FIG. 8A;

FIG. 8C is a Kaplan-Meier diagram indicating the survival of mice by defined end point criteria tor a period of 70 days after cell challenge treated with DACC/siRNA^{CD31}DACC/siRNA^{Luciferace} or 270 mM sucrose as control in an experimental lung metastasis mouse model as depicted in the scheme in FIG. 8A;

FIG. 8D is a diagram showing the inhibition of CD31 mRNA relative to the inhibition of CD34 mRNA in lung tissue of mice treated with DACC/siRNA^{CD31} lipoplexes, control lipoplexes or sucrose;

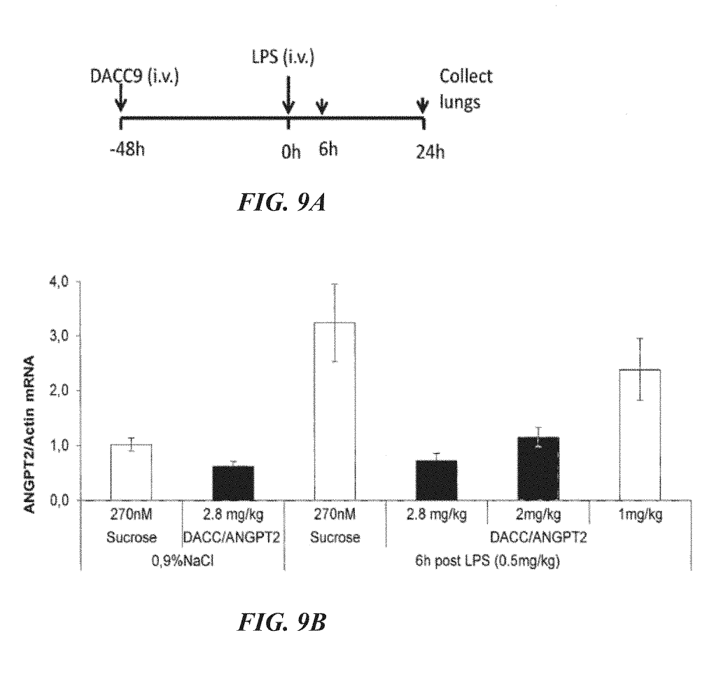

FIG. 9A is a representation of an experimental set-up for induction of an inflammatory response by LPS treatment and a treatment scheme using DACC/siRNA^{ANGPT2};

FIG. 9B is a diagram showing the inhibition of ANGPT2 mRNA relative to the expression of actin mRNA in lungs of mice 5 hours after challenge with either LPS (0.5 mg/kg) or saline (0.9 % NaC). Mice were treated with DACC/siRNA^{ANGPT2} lipoplexes with a dose of 2.8 mg/kg, 2.0 mg/kg and “10 mg/kg: 270 mM sucrose was used as a vehicle control;

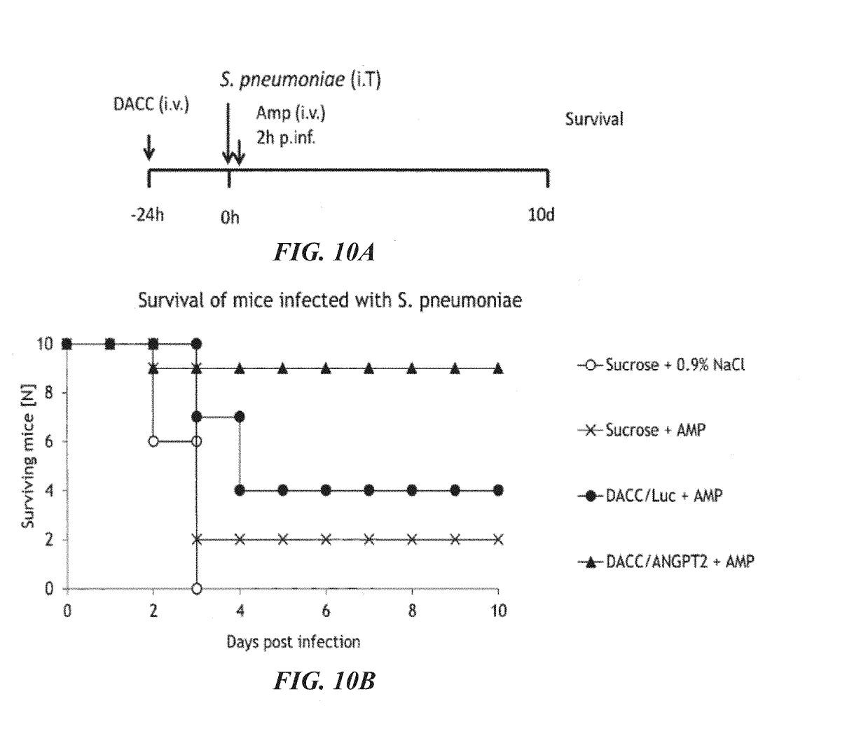

FIG. 10A is a representation of an experimental set-up for a S. pneumoniae infected lung mouse model and a treatment scheme using DACC/siRNA^{ANGPT2};

FIG. 10B is a Kaplan-Meier diagram indicating the survival of mice for a period of 10 days after challenge with S. pneumonia treated either with DACC/siRNA^{ANGPT2} and ampicillin, DACC/siRNA^{Luciferase} and ampicillin, sucrose and ampicillin or sucrose and vehicle (saline) as depicted in the scheme in FIG. 10A;

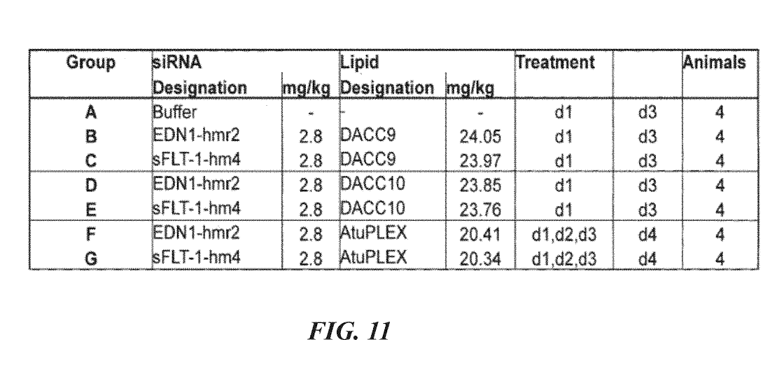

FIG. 11 is a summary outlining the experimental set-up for determining reduction of EDN1 expression in mice using different siRNA molecules targeting either EDN1 or soluble VEGF receptor 1 (sFit1) and different delivery systems; and

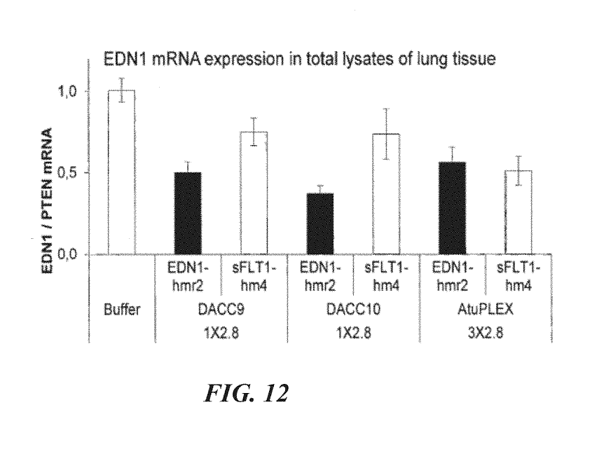

FIG. 12 is a bar diagram showing EDNI mRNA expression in total lysates of lung tissue from mice treated according to the experimental set-up illustrated in FIG. 5; EDN1 mRNA expression is normalized relative to PTEN mRNA.

### EXAMPLE 1

### Materials and Methods

The materials and methods described in the following were used throughout the examples, if not indicated to the contrary.

### Short Interfering RNAs

The siRNA molecules (AtuRNAi) used in the experiments subject to Examples 2 to 9 are listed in Table 1. The siRNA molecules (AtuRNAi) used in said Examples 2 to 9 are blunt, 19-mer double-stranded RNA oligonucleotides stabilized by alternating 2′-O-methyl modifications on both strands, and the siRNA molecules used in the experiments subject to Examples 10 to 11 are listed in Table 2. The siRNA molecules used in said Examples 10 to 11are blunt, 19-mer double-stranded RNA oligonucleotides stabilized by alternating 2′-O-methyl modifications on both strands with the control siRNA molecules targeting Luciferase are 23-mer double-stranded RNA oligonucleotides stabilized by alternating 2′-O-methyl modifications on both strands. Such modification has been previously described Santel A et al. (Santel, A el al. (2006). Gene Ther 13:1222-1234) and Czauderna, F. et al. (Czauderna, F. et al. (2003), Nucleic Acids Res 31: 2705-2716), and were synthesized by BioSpring (Frankfurt a.M., Germany), 2′-O-methyl modification of a nucleotide is indicated throughout this specification and the claims by the thus modified nucleotide being represented by a corresponding capital letter, whereas a non-modified nucleotide is represented by a corresponding lower ease letter.

TABLE 1

| siRNA | | |
|---|---|---|
| target | passenger strand (s) | guide strand (as) |
| PKN3 | 5′ aGaCuUgAgGaCuUcCu | 5′ UuGuCcAgGaAgUc |
| | GgAcAa 3′ | CuCaAgUcU 3′ |
| | (SEQ ID NO: 4) | (SEQ ID NO: 3) |
| siRNA- | 5′ cAgUaUcAgAaUuUcAg | 5′ AuCuUgCuGaAaUu |
| Cy3 | CaAgAu 3′ | CuGaUaCuG-CY3 |
| | (SEQ ID NO: 6) | (SEQ ID NO: 5) |
| Luci- | 5′ aUcAcGuAcGcGgAaUa | 5′ UcGaAgUaUuCcGc |
| ferase | CuUcGa 3′ | GuAcGuGaU 3′ |
| | (SEQ ID NO: 8) | (SEQ ID NO: 7) |
| Tie2 | 5′ cCaUcAuUuGcCcAgAu | 5′ AuAuCuGgGcAaAu |
| | Au 3′ | GaUgG 3′ |
| | (SEQ ID NO: 10) | (SEQ ID NO: 9) |
| CD31 | 5′ cAgAuAcUcUaGaAcGg | 5′ UuCcGuUcUaGaGu |
| | Aa 3′ | AuCuG 3′ |
| | (SEQ ID NO: 12) | (SEQ ID NO: 11) |
| KDR/ | 5′ cGgGgCaAgAgAaAuGa | 5′ AcAaAuUcAuUuCu |
| VEGFR2 | AuUuGu 3′ | CuUgCcCcG 3′ |
| | (SEQ ID NO: 14) | (SEQ ID NO: 13) |
| CDH5/VE- | 5′ uUgAaGcAaCuGuGaAu | 5′ GaAuUcAcAgUuGc |
| Cadherin | Uc 3′ | UuCaA 3′ |
| | (SEQ ID NO: 16) | (SEQ ID NO: 15) |
| BMPR2 | 5′ uUgCcAaGaUgAaUaCa | 5′ AuUgAuUgUaUuCa |
| | AucaAu 3′ | UcUuGgCaA 3′ |
| | (SEQ ID NO: 18) | (SEQ ID NO: 17) |

TABLE 2

| siRNA | passenger | guide |
|---|---|---|
| target | strand (s) | strans (as) |
| (Internal | (internal | (internal |
| reference) | reference) | reference) |
| ANGPT2 | 5′ gCaUgUgGuCcUu | 5′ AgUuGgAaGgAcCa |
| | CcAaCu 3′ | CaUgC 3′ |
| | (SEQ ID NO: 2) | (SEQ ID NO: 1) |
| | (ANGPT2-hmrc-3B) | (ANGPT2-hmrc-7A) |

Preparation and Characterization of siRNA Lipoplexes

Atnplex is a lipid composition containing
a) 50 mol % β-(L-arginyl)-2,3-L-diaminopropionic acid-N-palmityl-N-oleyl-amide tri-hydrochloride);
b) 49 mol % 1,2-diphytanoyl-sn-glycero-3-phosphoethanolamine (DPhyPE); and
c) 1 mol % N-(Carbonyl-methoxypolyethyleneglycol-2000)-1,2-distearoyl-sn-glycero-3-phosphoethanolamine sodium salt.

DACC9 is a lipid composition containing
a) 70 mol % β-(L-arginyl)-2,3L-diaminopropionic acid-N-palmityl-N-oleyl-amide tri-hydrochloride of the following formula:

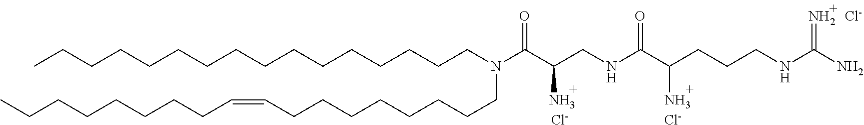 [SMILES: [Cl-] ; CCCCCCCC/C=C\CCCCCCCCN(CCCCCCCCCCCCCCCC)C(=O)[C@H](N)CNC(=O)C(N)CCCNC(=N)N ; [Cl-] ; [Cl-]]

b) 29 mol % cholesterol: and
c) 1 mol % mPEG-2000-DSPE of the following formula:

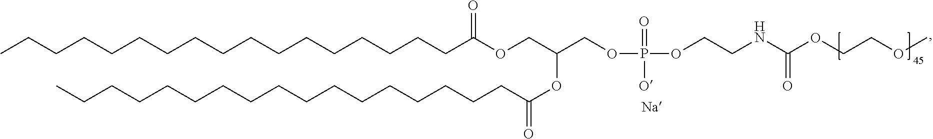

whereby the charge ratio [lipids/phosphate oligo] is 8.4.
DACC10 is a lipid composition containing
a) 70 mol % β-(L-arginyl)-2-3-L-diaminopropionic acid-N-palmityl-N-oleyl-amide tri-hydrochloride of the following formula:

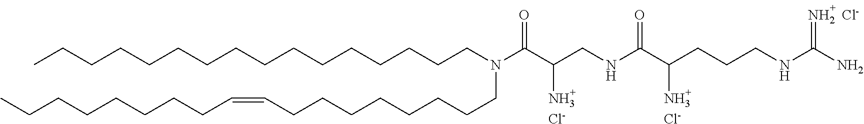 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]

b) 29 mol % cholesterol; and
c) 1 mol % mPEG-2000-Ceramide-C8 of the following formula:

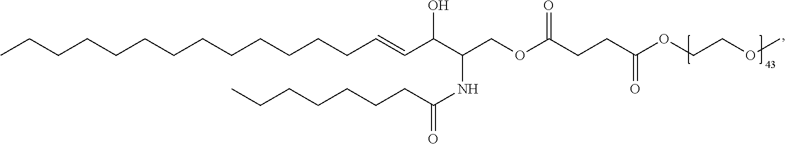

whereby the charge ratio [lipids/phosphate oligo] is 8.4.

Cationic liposomes, also referred to as DACC9, comprised of cationic lipid AtuFECT01 (β-L-arginyl-2,3 -L-diaminopropionic acid-N-palmityl-N-oleyl-amide trihydrochloride; Silence Therapeutics A G, Berlin, Germany), cholesterol (Sigma Aldrich) and mPEG2000-DSPE (1,2-distearoyl-sn-glycero-3phosphoethanol amine-N [methoxy (polyethylene glycol)-2000]); Avanti Polar Lipids Inc., Alabaster, Ala., USA) at a molar ratio of 70:29:1 were prepared by lipid film rehydration (Santel, A et al. (2006). Gene Ther 13: 1222-1234)) with 270 mM sterile sucrose solution. Cationic liposomes comprised of the above DACC10 lipid composition were prepared in an analogous manner. The resulting liposomal stock solutions had total lipid concentrations of 5 mg/ml or up to 9 mg/ml (e.g. for infusion studies), respectively. The formation of siRNA lipoplexes occurred by mixing equal volumes of liposomal dispersion and siRNA solution in 270 mM sucrose. For this purpose, the concentration of both were adjusted in a way, that the final lipoplex formulation was characterized by a final lipid/siRNA ratio [m/m] of ea. 6.8, which corresponded to a charge ratio between nucleic acid backbone phosphates to charged lipid nitrogen atoms (N/P ratio) of approximately 8.4. Particle sizes (Z-average size, intensity distribution) and seta potentials of liposomes and lipoplexes were determined by Dynamic Light Scattering using a Zetasizer Nano-ZS (Malvern Instruments, Worcestershire, UK). Here, corresponding dispersant properties were adjusted so 270 mM sucrose. Negative stain transmission electron microscopy (nsTEM) was done by Vironova AB (Stockholm, Sweden).

If reference is made to DACC, this means that such reference refers to both DACC9 and DACC10.

siRNA Distribution and RNAi In Vivo

Eight week old C57B1/6j (Harlan) males and females were used for in vivo studies. All animal experiments in this study were performed according to approved protocols and in compliance with the guidelines of the Landesamt für Arbeiss-, Gesundheissschutz und technisehe Sicherneit Berlin, Germany (No. G0264/99). Isotonic siRNA lipoplex formulations were administered intravenously through bolus tail vein injection at the indicated doses. For siRNA-Cy3 biodistribution studies, mice received a single dose of DACC9siRNA-Cy3 (2.8 mg siRNA/kg body weight), 1 hour post-injection mice were sacrificed by cervical dislocation and tissues were processed for paraffin embedding as previously described (Santel, A et al. (2006). Gene Ther 13: 1222-1234)). Sections of 4 μm were cut and deparaffinized with Roticlear (Roth, A538..5), rehydrated through graded ethanol washes and counterstained with Sytox Green dye (Molecular probes). Fluorescence uptake was analyzed with a Zeiss LSM510 Meta confocal microscope. Images were recorded and processed by Zeiss LSM5 software. For quantitative analysis of siRNA distribution in different organs, mice received a single dose of lipoplexes formulated with PKN3 siRNA. Concentrations of PKN3 siRNA in different tissues were determined by a modified capture probe sandwich hybridization assay (Aleku, M, et al. (2008). Cancer Res 68; 9788-9798)/. For target mRNA knock down analyses, tissues were dissected immediately after sacrifice of the mice and instantly snap-frozen in liquid nitrogen. Approximately 20 mg of tissue was homogenized in a Mixer Mill MM 301 (Retsch GmbH, Haan, Germany) using tungsten carbide beads (Qiagen). Total RNA was isolated from the lysate with the Invisorb Spin Tissue RNA Mini Kit (Invitek, Berlin, Germany). Depending on the tissue, 25-100 ng total RNA was used tor quantitative TaqMan RT-PCR with the amplicon sets (listed in Table 3) obtained from BioTez GmBH, Berlin, Germany): The TaqMan RT-PCR reactions were carried out with an ABI PRISM 7700 Sequence Detector (Software: Sequence Detection System v 1.6.3 (ABI)) or StepOnePlus™ Real Time PGR Sytem (ABI) using a standard protocol for RT-PCR as described previously (Santel, A et al. (2006). Gene Ther 13: 1222-1234)) with primers and probes at a concentration of 300 and 100 nM respectively. TaqMan data were calculated by using the Comparative CT method. Target protein expression was assessed by Western blotting of whole tissue lysates as described previously (Santel. A et al (2006). Gene Ther 13: 1222-1234)). Snap frozen tissues were homogenized in a Mixer Mill MM 301 (Retsch GmbH, Haan, Germany), and proteins were extracted in Riper-lysis buffer. Equal amounts of protein were loaded for immunobot analysis using the following antibodies: rabbit anti-PTEN (Ab-2, Neomarkers, Fremont, Calif., USA) and mouse anti-Tie2 (clone Ab33, Upstate 05-584).

TABLE 3
Primer sets for Taqman analysis

| Target | Upper | Lower | |
|---|---|---|---|
| Genes | Primer | primer | Probe |
| Tie-2 | ATGAGACAGTGCT | AGCATCCTGTTT | AGCCTTATGAGAGAC |
| | GGAGGGAGA | AAGGACACCAA | CATCATTTGCCCAG |
| | (SEQ ID | (SEQ ID | (SEQ ID |
| | NO: 19) | NO: 20) | NO: 21) |
| CD31 | GGGAACGAGAGC | CATTAAGGGAGC | CGGAAGGTCGACCCT |
| | CACAGAGAC | CTTCCGTTC | AATCTCATGGAAA |
| | (SEQ ID | (SEQ ID | (SEQ ID |
| | NO: 22) | NO: 23) | NO: 24) |
| VEGF-R2 | TCAATGGAGAAAC | GAATCCATAGG | TGGTCTCTCTGGTTG |
| | AGAGCCACA | CGAGATCAAGG | TGAATGTCCCACC |
| | (SEQ ID | (SEQ ID | (SEQ ID |
| | NO: 25) | NO: 26) | NO: 27) |
| BMPR2 | CCGTCTGGCTCAT | GGGAGATTGCG | TGGCTTATCTTCACAC |
| | TCTGTGA | GGTTTATAATG | AGAATTACCACGAGG |
| | (SEQ ID | (SEQ ID | (SEQ ID |
| | NO: 28) | NO: 29) | NO: 30) |
| VE- | GAACGAGGACAG | CTCCCGATTAA | TCATAAACAACCATGA |
| cadherin | CAACTTCACC | ACTGCCCATAC | CAACACCGCCAA |
| | (SEQ ID | (SEQ ID | (SEQ ID |
| | NO: 31) | NO: 32) | NO: 33) |
| PTEN | CACCGCCAAATTT | AAGGGTTTGATA | TGCACAGTATCCTTTT |
| | AACTGCAGA | AGTTCTAGCTGT | GAAGACCATAACCCA |
| | (SEQ ID | (SEQ ID | (SEQ ID |
| | NO: 34) | NO: 35) | NO: 36) |
| Actin | GTTTGAGACCTTC | GACCAGAGGCA | CCATGTACGTAGCCA |
| | AACACCCCA | TACAGGGACA | TCCAGGCTGTG |
| | (SEQ ID | (SEQ ID | (SEQ ID |
| | NO: 37) | NO: 38) | NO: 39) |
| CD34 | GAGGCTGATGCT | CAGCAAACACT | CTGCTCCCTGCTTCTA |
| | GGTGCTAGT | CAGGCCTAACC | GCCCAGTCTGA |
| | (SEQ ID | (SEQ ID | (SEQ ID |
| | NO: 40) | NO: 41) | NO: 42) |
| ANGPT2 | CAAAGGATTCGGA | CATTCAAGTTGG | TCCCAGATGCTCTCA |
| | CAATGACAA | AAGGACCAC | GGAGGCTGGTGGT |
| | (SEQ ID | (SEQ ID | (SEQ ID |
| | NO: 43) | NO: 44) | NO: 45) |

### Infusion Studies

For infusion studies jugular vein catheterized mice (Harlan) received a single 1 hour infusion of DACC9/lipoplexes of the highest dose (12 mg siRNA/kg body weight; 40 ml/kg body weight). For dose titrations, DACC9 lipoplex stock solution was diluted in 5% Glucose solution to keep the administration volume constant.

### Transfection of Mouse Endothelial Cell Line, MS1

The mouse endothelial cell line MS1(ATCC CRL-2279) was obtained from the American type cell culture collection (ATCC) and cultivated according to the supplier's recommendations. Cells were seeded in 6 well plates and transfected with DACC9/siRNATie-2 as described previously (Samel, A et al. (2006), Gene Ther 13: 1222-1234)). Briefly, about 12 hours after cell seeding different amounts of DACC9/siRNA formulations diluted in 10% serum-containing medium were added to the cells to achieve transfection concentrations in a range of 10 to 160 nM siRNA. Three days alter transfection cells were lysed. Proteins were separated by SDS-PAGE and subjected to immunoblotting as previously described (Santel, A et al. (2006). Gene Ther 13: 1222-1234)).

### Experimental Lung Metastasis Mouse Model

Lewis lung carcinoma (LLC) cells were cultured in RFMI medium supplemented with 10 % FCS and 4 mM glutamine. Cells were dissociated with trypsin and subsequently washed in cell culture medium and in PBS. 250 000 cells in 200 μl PBS were injected into the tail vein of 8 week old male BDF1 mice (Harlan). Mice were treated 11 times with isotonic sucrose or with DACC9 lipoplexes on alternating days beginning 5 days before tumor cell challenge.

Mice were monitored daily for bodyweight development and signs of suffering. When reaching defined endpoint criteria (score >=4) animals were sacrificed by cervical dislocation. Endpoint criteria were signs of suffering (cachexia, weakening, difficulty moving or eating, compound toxicity (hunching convulsions), 15% reduction of body weight for 3 consecutive days or 26% body weight loss for 1 day and a score of >=4.)

Streptococcus pneumonia Infected Lung Model

The study was conducted by Eurofin/Panlabs (Ricerca, study B09784, adapted from model 608100): Male specific pathogen free ICR mice weighing 20 to 22g were treated with DACC9 lipoplex (2.8 mg siRNA/kg body weight) or sucrose as vehicle control by tail vein injection; 24 hours later, all mice were infected intratracheally with Streptococcus pneumoniae (ATCC 6301) with LD 90-100 dose (0.02 ml, 7-9×10⁶ CPU) to induce acute pneumonia. 2 hours after infection a single suboptimal dose of Ampicillin (3 mg/kg) was given by intravenous route. Survival of mice is monitored up to 10 days after infection. Without treatment with ampicillin, mice died within 3 days after infection with S. pneumonia. Only a moderate increase in survival was observed by single dose of ampicillin. DACC9/Angpt2 siRNA pretreatment in addition to Ampicillin treatment enhances survival of mice significantly. Survival of >50% of animals indicates significant activity of test substance.

### Statistical Analysis

Data are expressed as means ±SEM.

### EXAMPLE 2

### Lung Specificity of DACC

Different lipoplex formulations were evaluated in vivo in order to investigate their respective capacity to deliver siRNA cargo to select organs. All lipoplexes used to examine siRNA biodistribution pattern were formulated with the cationic lipid AtuFECT01 and siRNA^{PKN-3} but contained different co-lipids and/or PEG-lipids at different ratios. Lipoplexes were administered intravenously via tail-vein injection, and siRNA concentrations were determined in liver, kidney, lung, heart and spleen tissue samples 1 hour alter systemic application using a siRNA specific quantitative ELISA-based capture-probe assay The results are shown in FIG. 1.

The composition of the various lipoplcxcs is summarized in Table 4.

TABLE 4
Composition ofvarious lipoplexes

| Lipoplex | cationic lipid | co-lipid | co-lipid | PEG-lipid | molar ratio [%] |
|---|---|---|---|---|---|
| lipoplex_01 | 16/18:1DapArg | | | | 100 |
| lipoplex_02 | 16/18:1DapArg | DPhyPE | | mPEG2000-DSPE | 50:49:01 |
| lipoplex_03 | 16/18:1DapArg | Cholesterol | | mPEG2000-DSPE | 50:49:01 |
| lipoplex_04 | 12/14DapArg | Cholesterol | | mPEG2000-DSPE | 70:29:01 |
| DACC9 | 16/18:1DapArg | Cholesterol | | mPEG2000-DSPE | 70:29:01 |
| lipoplex_05 | 16/18:1DapArg | CHEMS | | mPEG2000-DSPE | 70:29:01 |
| lipoplex_06 | 16/18:1DapArg | DOPC | | mPEG2000-DSPE | 25:74:1 |
| lipoplex_07 | 16/18:1DapArg | DOPC | | mPEG2000-DSPE | 50:49:01 |
| lipoplex_08 | 16/18:1DapArg | DOPC | | mPEG2000-DSPE | 75:24:01 |
| lipoplex_09 | 16/18:1DapArg | Cholesterol | CHEMS | mPEG2000-DMPE | 19:30:59:2 |
| lipoplex_10 | 16/18:1DapArg | Cholesterol | CHEMS | mPEG2000-DMPE | 45:33:20:2 |

Concentrations of siRNA delivered to the respective tissues varied depending on the lipoplex system used, whereas the DACC9 lipoplex system displayed the most efficient siRNA delivery to the lungs (FIG. 1, black bar). Furthermore, an inverse correlation between lipoplex formulation stability and functional organ uptake, i.e. the formulations most stable even with respect to their tendency to aggregate were also found to be least entrapped in the vascular beds of the pulmonary endothelium, but rather to accumulate directly in the liver and spleen for subsequent degradation. However, delivery of siRNA to the target tissue and cellular uptake were not the sole factors responsible for activity as demonstrated previously by Heyes et al. (Heyes, J, Palmer, L, Bremner, K, and MacLachlan, I (2005), J Control Release 107: 276-287) for a different cationic lipid based system, suggesting that instead of endocytosis being rate-limiting, it is rather those events occurring once the siRNA has been internalized by the cell which have the greatest effect on the efficiency of gene-silencing, such as endosomal release and RISC loading.

### EXAMPLE 3

### Lipid Composition and Physico-Chemical Characterization of the DACC/siRNA Lipoplexes

DACC9 lipoplexes, sometimes also referred to as DACC9 lipoplexes herein, are composed of the positively charged lipid system AtuFECT01 (β-L-arginyl-2,3-L-diaminopropionic acid-N-palmityl-N-oleyl-amide trihydrochloride), cholesterol and mPEG2000-DSPE (1,2-distearoyl-sn-glycero-3-phosphoethanol amine-N [methoxy (polyethylene glycol)2000]) in a molar ratio of 70:29:1 together with a blunt ended siRNA duplexes chemically stabilized by alternating 2′-Q-methyl modifications on both strands (Santel, A, et al. (2006), Gene Ther 13; 1222-1234; Aleku, M. et al. (2008), Cancer Res 68: 9788-9798) in 270 mM sucrose solution. The chemical compounds of DACC9, their chemical structure and their molar ratio, expressed in percent, as well as the basic design of the siRNA molecules of the lipoplexes are indicated in FIG. 2A. In connection with the siRNA molecules it is to be noted that they are blunt-ended and that the circles indicate a 2-O-methyl modified nucleotide.

The resulting DACC9 lipoplex particles were characterized regarding size and zeta potential. The Z-average size amounted to ˜70 nm as determined by dynamic light scattering in a 270 mM sucrose solution with the result being shown in FIG. 2B, and the zeta potential measured in 270 mM sucrose was between 40 and 50 mV as may be taken, from FIG. 2C.

Electron microscopy of DACC9 lipoplex particles revealed predominantly lamellar structures in mostly spherical arrangements as shown in FIG. 2D. The addition of sucrose enables formulation stability during freezing, drying and rehydration steps, thereby ensuring effective long-term storage as a lyophilized product (data not shown). Similar results were obtained for DACC10 lipoplexes composed of the DACC10 lipid composition a blunt ended siRNA duplexes chemically stabilized by alternating 2′-O-methyl modifications on both strands (Santel, A, et al. (2006), Gene Ther 13:1222-1234; Aleku, M, et al. (2008), Cancer Res 68: 9788-9798) in 270 mM sucrose solution.

### EXAMPLE 4

### Primary Delivery of siRNAs to Lung Endothelium by DACC

To characterize the tissue distribution and kinetics of siRNA delivery by systemic application of DACC9 lipoplexes in more detail, mice were treated with a single dose of DACC9 (2.8 mg siRNA/kg body weight) and a number of tissue samples from different organs were collected 1, 6 and 24 hours after administration of the DACC9 lipoplex formulation to determine respective siRNA concentrations. The results are shown in FIG. 3A.

As may be taken from FIG. 3A, highest siRNA concentrations [about 40% ID/g tissue] were found in lung tissue at the 1-hour time point, followed by spleen, liver and to lesser extent kidney and heart, siRNA concentrations in all tissues investigated diminished over time, siRNA levels below 1% of initial dose were measured in blood, brain, prostate or skeletal muscle.

In order to examine siRNA distribution within predominantly targeted tissue, mice were treated with DACC9 lipoplexes formulated with cyanine dye (Cy3) labeled siRNA. Cy3-labeled siRNAs in the tissues were then visualized by confocal microscopy of formalin-fixed paraffin embedded tissue sections, siRNA distribution patterns in lung, liver, kidney and heart were reminiscent of vascular staining patterns as may be taken from FIGS. 3b-c.

From all organs investigated, lungs were most intensely stained by siRNA-Cy3 where siRNA staining is evenly distributed in the lung vasculature.

Upon close up-view, siRNA derived staining in the lungs was finely dotted and centered around cell nuclei, this observation being indicative for intracellular uptake of siRNAs (FIG. 3B). In the heart, distinct Cy3 staining was found lining the capillaries, while muscle fibers were tree of siRNA-Cy3 signal (FIG. 3C). The sinusoidal endothelial cell layer in the liver showed weak siRNA-Cy3 staining pattern (FIG. 3C), while individual cells within the liver sinusoids were strongly Cy3 stained. The oval shaped nucleus (FIG. 3C, see arrow) marks Kupffer cells responsible for the removal of foreign particulate substances, hence, lipoplexes found here could also have been taken up by phagocytosis (Whitehead, K A et al. (2009), Nat Rev Drug Discov 8:129-138). Hepatocytes, discernible by their large, regular and round nucleus, were free of siRNA-Cy3staining. siRNA-Cy3 staining in the spleen was pronounced in the marginal zones of the white pulp, while the center of the white pulp remained CY3- staining free (FIG. 3C). Since monocytes and macrophages known to be responsible for lipoplex clearance from the blood are sequestered into this zone, lipoplex clearance by respective macrophages could explain the enhanced siRNA-Cy3 staining patterns observed in this area. Distinct Cy3 signals were also detected in peritubular capillaries of the kidney (FIG. 3C, arrow). Staining of tubular cells was diffuse, with a tendency for Cy3-siRNA accumulation towards the lumen of the tubuli, indicative for secretion of free siRNA by the kidney. Altogether, the quantitative and qualitative analysis of siRNA distribution by the DACC9 delivery system revealed siRNA uptake to occur mainly in the lungs, but distinct siRNA-Cy3 derived signals were also detected in the microvasculature of heart, liver and kidney as well as in phagocytic cells of liver and spleen.

### EXAMPLE 5

### Inhibition of Target Gene Expression by DACC9 in Lung Vasculature

To test whether siRNA delivered by DACC9 is functionally active and can be used for inhibition of target gene expression in vascular beds, DACC formulations with siRNAs specific for the target gene Tie-2 were prepared. Tie-2 expression is highly specific for endothelial cells and it is a common marker for this cell type in many organs (van der Heijden, M et al., Expert Opin Ther Targets 13:39-53). RNAi activity of DACC/siRNA^{Tie-2} was first tested in vitro. Human umbilical vein endothelial cells (HUVECs) were transfected with DACC9MRNA^{Tie-2}, and Tie-2 protein expression was assessed in cell lysates by immuno-blotting after 72 hours. The results are shown in FIG. 4A.

Tie-2 expression was significantly reduced by DACC9/siRNA^{Tie-2} at a dose of 160 and 80 nM siRNA, and to a lesser extent 40 nM siRNA, (FIG. 4A), which confirmed that DACC9can functionally deliver siRNA into cells and mediate RNAi. It should be noted that these concentrations are not reflective of the siRNAs IC50s, since DACC9 is not optimized for cell culture transaction experiments, and lower concentrations can be used in the latter.

To investigate whether DACC9 is capable of gene silencing in the vascular endothelium in vivo. mice were given three doses of DACC9 lipoplexes on consecutive days by tail vein injection (3×2.8 mg/kg). Lung, heart, liver, and kidney tissues were collected 24 hours after the last treatment. Total mRNA was prepared from whole tissue lysates, and target mRNA levels were assessed by quantitative RT-PCR. The results are shown in FIGS. 4B-E. Tie-2 mRNA levels were reduced by over 80 % in the lung tissue of mice treated systemically with DACC9/siRNA^{Tie-2} as compared to control treatments with either DACC9/siRNA^{control} or with sucrose solution (FIG. 4B). No significant Tie-2 knock-down was observed in liver, kidney, and heart tissue even after repeated dosing of DACC9/siRNA^{Tie-2} formulation (FIGS. 4C-E), indicating that the lungs are the primary organ functionally targeted by DACC9/siRNA^{Tie-2}.

### EXAMPLE 6

### DACC/siRNA Dose Responsiveness of Target Gene Expression

To further investigate the dose requirements for target gene inhibition by DACC9 lipoplexes in mice, single doses of 3.0, 1.5 and 0.75 mg siRNA/kg body weight of DACC9/siRNA^{Tie-2} or DACC9siRNA^{CD31} were administered by intravenous tail-vein bolus injection. Lung tissue was collected 24 hours post injection for RNA analysis. The results for inhibition of Tie-2 target gene expression are shown in FIG. 5A.

Surprisingly, already a single dose of DACC9/siRNA^{Tie-2} (3.0 mg siRNA/kg) injected by bolus treatment sufficed to reduce Tie-2 mRNA levels similar to those obtained after repeated dosing as described for FIG. 4B (3×2.8 mg siRNA/kg) (compare, FIGS. 5A and 4B). 1.5 mg siRNA/kg was also effective in reducing Tie-2 expression, but to a lesser extent, and 0.75 mg siRNA/kg did not affect Tie-2 levels significantly. Since all DACC9 applications described so far were performed by bolus injection, but application via infusion is the preferred mode of lipoplex delivery in humans, different doses of DACC9/siRNA^{Tie-2} were administered into the jugular vein of mice by 1 hour infusions. Such administration by mode of infusion (the infusion is administered over a period of time of about 1 h), is a prerequisite in clinical settings. The results are shown in FIG. 5B.

Target gene silencing after DACC9/siRNA^{Tie-2} infusion was comparable to that by bolus injection at 3 mg siRNA/kg body weight. Tie-2 mRNA levels in lung tissue were reduced by approximately 80%. Since this mode of application enabled the use of larger volumes of DACC9 lipoplexes as compared to bolus injection, doses administered were increased to 6 mg siRNA/kg and/or 12 mg siRNA/kg, respectively. This increase in lipoplex concentration was shown to reduce Tie-2 expression levels even further to over 95% (FIG. 5B). Nonetheless, all animals tolerated infusion treatment of DACC9/siRNA^{Tie-2} even at highest dosage (12 mg/kg)(FIG. 5B).

Based on the above data an EC50 in the mouse of about 1.5 mg siRNA/kg for inhibition of Tie-2 expression in the lungs using a single dose, but higher doses of 3 mg/kg (bolus) and up to 6 mg/kg (infusion) were also tolerated. However, higher single doses of siRNA lipoplexes had unfavorable effects on body weight development, showing a drop of over 10 % at the 12 mg siRNA/kg dose levels (compared to negligible weight loss at a dose of 3 and 1 mg siRNA/kg (data not shown). From a clinical standpoint, the observed low and one-time dosing is favorable for subsequent toxicity profiling.

### EXAMPLE 7

### Duration of Target Inhibition and Verification of RNAi-Mediated Knock-Down Mediated by DACC

To assess duration of target gene inhibition by DACC9 lipoplexes, mice were treated with a single dose (2.8 mg/kg) of DACC9/siRNA^{Tie-2} or DACC9/siRNA^{ANGPT2} and cohorts were sacrificed 3, 7, 14 and 21 days post treatment (p.t.). Tie-2 target mRNA levels in the respective lung tissue were quantified by quantitative RT-PCR. The results are shown in FIG. 6A.

Most prominent reduction of Tie-2 expression in the DACC9/siRNA^{Tie-2} treatment group was observed three days p.t. with lipoplexes (over 80 % Tie-2 reduction compared to DACC9control groups). Nonetheless, Tie-2 mRNA levels were still reduced by over 50% (FIG. 6A) after 21 days, as compared to control groups treated either with sucrose or with control DACC9 lipoplex. Remarkably, a single dose of DACC9 lipoplex (2.8 mg siRNA/kg) was shown to reduce mRNA levels of the target gene Tie-2 by 60-90 % (FIG. 6A), with this silencing effect persisting for up to three weeks.

To confirm that the reduction in Tie-2 mRNA leads to a corresponding reduction of Tie-2 protein, lung tissue homogenates were prepared from mice 3 and 21 days after treatment with DACC9 lipoplex or with sucrose as vehicle control, respectively. Tie-2 protein was detected by Western blotting as shown in FIG. 6B. Most prominent reduction of Tie-2 protein levels were seen three days after treatment, corresponding to the respective decrease of Tie-2 mRNA levels, and were shown to gradually subside over time. However a profound reduction of Tie-2 protein expression was still observed 21 days after treatment. Furthermore, Tie-2 mRNA levels remain reduced by over 70 % after 21 days.

### EXAMPLE 8

### Inhibition of Additional Target Genes in the Pulmonary Endothelium by DACC/siRNA

In order to confirm that the DACC9 delivery system is also capable of inhibiting other genes expressed in the pulmonary endothelium apart from Tie-2, DACC9 lipoplexes were prepared with specific siRNAs for other gene targets whose expression is highly restricted to endothelial cells: the VEGFR2 receptor, VE-cadherin, BMPR2 and the CD31 gene with the sequences of the respective siRNAs being as indicated in Example 1. Treating mice with a single injection (2.8 mg siRNA/kg) of DACC9/siRNA^{VEGFR2, DACC}9/siRNA^{VE-Cadherin}, DACC9/siRNA^{BMPR-2}, or DACC9/siRNA^{CD31} reduced mRNA levels of respective target genes by 60-90% as shown in FIG. 7. The observed reduction of mRNA levels thus demonstrates that DACC9 lipoplexes are capable of functionally delivering siRNA to the vascular endothelium, thereby enabling target-specific gene silencing in this tissue type. Furthermore, merely a single application of DACC9 lipoplexes was sufficient for down-regulating the expression of the respective target genes in lung vasculature.

### EXAMPLE 9

### DACC/siRNA^{CD31} Treatment Increases Survival in Experimental Lung Metastasis Mouse Model

CD31/Pecam 1 is a cell surface protein required for homotypic as well as heterotypic cell interactions. It is involved in multiple processes of tumorigenesis, like angiogenesis, vascular, permeability and metastases (Cao, G et al. (2009), Am J Pathol 175:903-915; Delisser, H et al. (2010), Proc Natl Acad Sci USA 107:18616-18621). It was also demonstrated in previous studies that targeting CD31 by RNA interference leads to reduction of tumor growth in subcutaneous xenograft tumor models and in orthotopic prostate cancer model (Santel, A, et al. (2006), Gene Ther 13:1360-1370).

In the instant study it was investigated whether DACC9/siRNA^{CD31} could be employed therapeutically in the treatment of lung cancer. To this end, it was tested whether treatment with DACC9/siRNA^{CD31} had a therapeutic benefit in an experimental lung metastasis mouse model (Santel, A et al. (2010), Clin Cancer Res 16:5469-5480). In this model, Lewis lung carcinoma cells (LL) are applied intravenously to syngeneic BDF1 mice, thus leading to colonization of tumor cells in the lungs and subsequent outgrowths of pulmonary metastases. DACC9/siRNA^{CD31}, control lipoplexes DACC9/siRNA^{luciferase}, and sucrose as vehicle control were prepared and injected into mice five days prior to LL tumor cell i.v injection. Treatment by bolus injection (2.8 mg/kg) was repeated on alternating days until day 15 (FIG. 8A).

Body weight was monitored continuously. Decrease in body weight due to DACC9 lipoplex application during the treatment period was not observed as may be taken from FIG. 8B.

Survival of animals was monitored by defined endpoint criteria for up to 70 days after tumor cell challenge. The results are shown in FIG. 8C. Survival of animals which received DACC9/siRNA^{CD31} was significantly enhanced as compared to control treatment group that received sucrose (p<0.006, log-rank test) or DACC9/siRNA^{luciferase} (p<0.001). Animals treated with isotonic sucrose solution or luciferase control lipoplexes showed poor survival: in the sucrose control group, none of the animals survived past 30 days, and only two animals of the DACC9/siRNA^{luciferase} control group survived up to day 70. In comparison, 7 of 8 animals receiving DACC9/siRNA^{CD31} survived up to day 70. CD31 expression was evaluated after completion of the treatment phase on day 16 after tumor cell challenge in separate cohorts (FIG. A). Compared to animals treated with DACC9/siRNA^{luciferase} or sucrose as vehicle control, CD31 expression in lungs of animals treated with DACC9/siRNA^{CD31} was reduced by approximately 80% (FIG. 8D) confirming that CD3 1 expression could be targeted by DACC9 lipoplexes in a therapeutic setting.

### EXAMPLE 10

### Prevention of Induction of ANG2 by LPS in mice by DACC/SiRNA^{ANG2}

C57BL6 mice were treated intravenously with the indicated doses of DACC9 lipoplex or with sucrose solution. 48 hours later, mice were challenged with LPS (0.5 mg/kg, i.v.) or saline (0.9% NaCl), Lung tissues were collected 6 hours after the LPS treatment and processed for RNA isolation (see FIG. 9A), ANGPT2 mRNA levels in lung tissue samples were determined by qRT-PCR. Actin mRNA levels were used as normalizer. Other aspects of the experiment related to materials and methods used, were carried out in accordance with Example 1.

The results are shown in FIG. 9B. As may be taken from FIG. 9B, the sucrose treatment groups LPS induced elevation of ANGFT2 mRNA levels in lung tissue. DACC9/ANGPT2 siRNA treatment reduced ANGPT2 induction in a dose dependent manner.

### EXAMPLE 11

### BACC/siRNA Treatment Increases Survival in S. pneumoniae Infected Long Model (Mouse)

Male specific pathogen free ICR mice weighing 20 to 22 g were treated with DACC9 lipoplex (2.8 mg siRNA/kg body weight) or sucrose as vehicle control tor the lipoplex by tail vein injection. 24 hours later, all mice were infected intratracheally with Streptococcus pneumoniae (ATCC 6301) with LD 90-100 dose (0.02 ml, 7-9×10⁶ CPU) to induce acute pneumonia. 2 hours after infection a single suboptimal dose of Ampicillin, “AMP”, (3mg/kg) or 0.9% NaCl was given by intravenous route. Survival of mice is monitored up to 10 days after infection (see FIG. 10A). Other aspects of the experiment related to materials and methods used, were carried out in accordance with Example 1. The results are shown in FIG. 10B.

As may be taken from FIG. 10B, without treatment with Ampicillin, mice died within 3 days after infection with S. pneumonia. Only a moderate increase in survival was observed by single dose of Ampicillin, DACC9/Angpt2 siRNA (DACC9/siRNA^{Angpt2}) pretreatment in addition to Ampicillin treatment enhances survival of mice significantly. Survival of >50% of animals indicates significant activity of test substance.

### EXAMPLE 12

### Reduction of EBNG Expression in Mice

The purpose of this animal study was to evaluate different delivery systems for targeting the endothelin 1 coding gene EDN1. The experimental set-up and treatment scheme for the mice is outlined in FIG. 11.

Mice were treated with a single dose of an EDN 1-targeting siRNA referred to as EDN1-hmr2(consisting of two separate strands which are 100% complementary to each other, whereby each strand consists of 19 nucletoides) formulated with DACC9, DACC10 or with three doses of EDN1-hmr2. siRNA formulated with Atuplex by bolus application. Control lipoplexes contained siRNA targeting the gene coding for soluble VEGF receptor 1 (sF1t1) which is referred to as sFLT1-hm4. Target gene expression was analyzed in long tissues 48 hours post treatment.

The results are shown in FIG. 12. From said FIG. 12 it is evident that for the purpose of delivery siRNA to lung tissue siRNA formulated with either DACC9 or DACC10 is more potent than siRNA formulated with Atuplex.

All of the references recite herein are herewith incorporated by reference.

The features of the present invention disclosed in the specification, the claims and/or the drawings may both separately and in any combination thereof be material for realizing the invention in various forms thereof.

1-121. (canceled)

122. A composition comprising a lipid composition, wherein the lipid composition consists of a cationic lipid of formula (I) 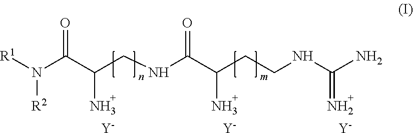
wherein n is any one of 1, 2, 3 and 4,
wherein m is any one of 1, 2 and 3,
Y⁻ is an anion,
wherein each of R1 and R2 is individually and independently selected from the group consisting of linear C12-C18 alkyl and linear C12-C18 alkenyl;
a sterol compound, wherein the sterol compound is selected from the group consisting of cholesterol and stigmasterol; and
a PEGylated lipid, wherein the PEGylated lipid comprises a PEG moiety and wherein the PEGylated lipid is selected from the group consisting of:
a PEGylated phosphoethanolamine of formula (II) 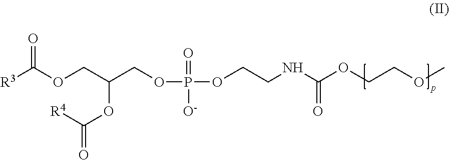
wherein each of R3 and R4 is individually and independently linear C13-C17 alkyl, and p is any integer from 15 to 130;
a PEGylated ceramide of formula (III) 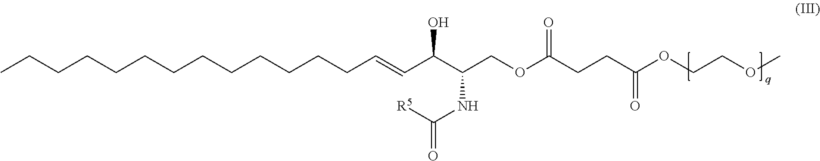
wherein R5 is linear C7-C15 alkyl, and
q is any integer from 15 to 130; and
a PEGylated diacylglycerol of formula (IV) 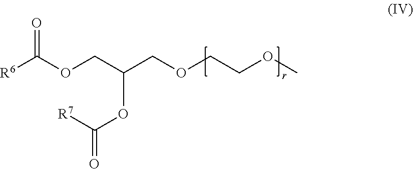
wherein each of R6 and R7 is individually and independently linear C11-C17 alkyl, and r is any integer from 15 to 130.

123. The composition according to claim 122, wherein the cationic lipid is selected from a compound of formula (Ia) 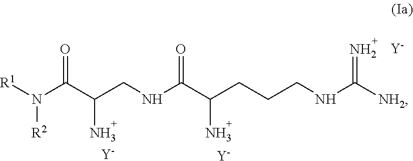
β-arginyl-2,3-diamino propionic acid-N-palmityl-N-oleyl-amide trihydrochloride of formula (Ib) 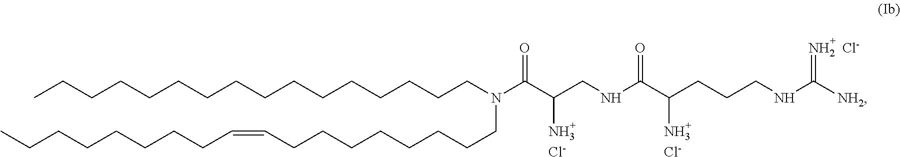 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]
β-arginyl-2,3-diamino propionic acid-N-lauryl-N-myristyl-amide trihydrochloride of formula (Ic) 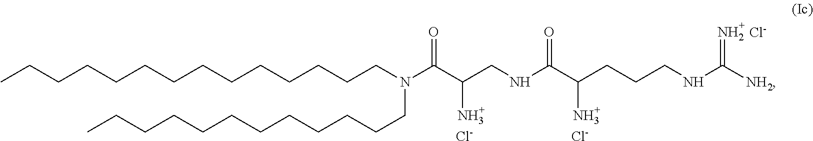 [SMILES: CCCCCCCCCCCCCCN(CCCCCCCCCCCC)C(=O)C(N)CNC(=O)C(N)CCCNC(=N)N ; [Cl-] ; [Cl-] ; [Cl-]]
or
ϵ-arginyl-lysine-N-lauryl-N-myristyl-amide trihydrochloride of formula (Id)

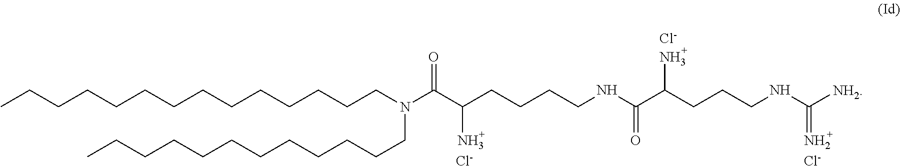 [SMILES: [Cl-] ; CCCCCCCCCCCCCCN(CCCCCCCCCCCC)C(=O)C(N)CCCCNC(=O)C(N)CCCNC(=N)N ; [Cl-] ; [Cl-]]

124. The composition according to claim 122, wherein the PEGylated phosphoethanolamine of formula (II) is selected from:

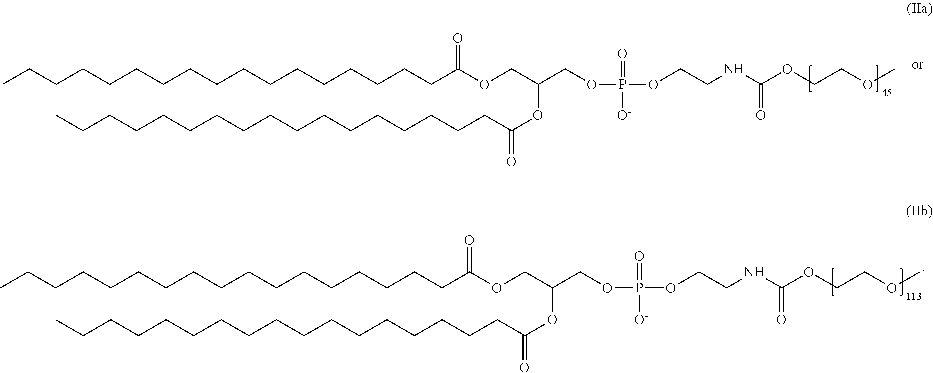

125. The composition according to claim 122, wherein the PEGylated ceramide of formula (III) is selected from:

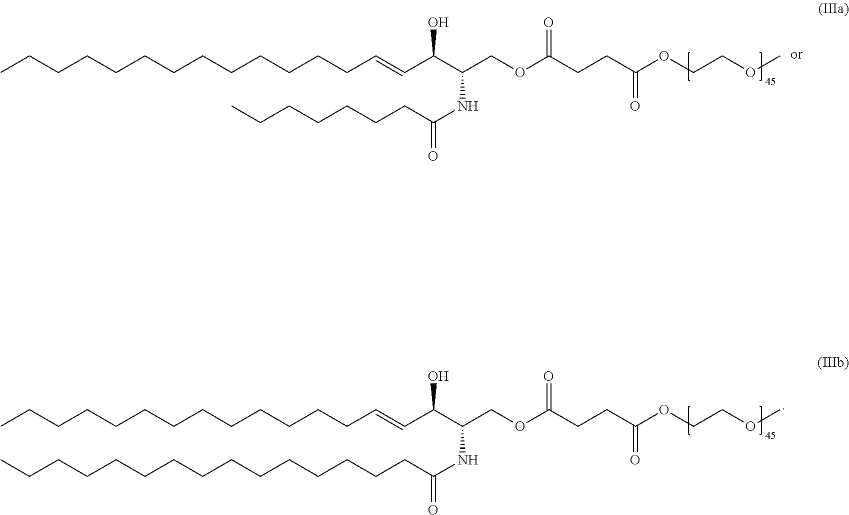

126. The composition according to claim 122, wherein the PEGylated diacylglycerol of formula (IV) is selected from:

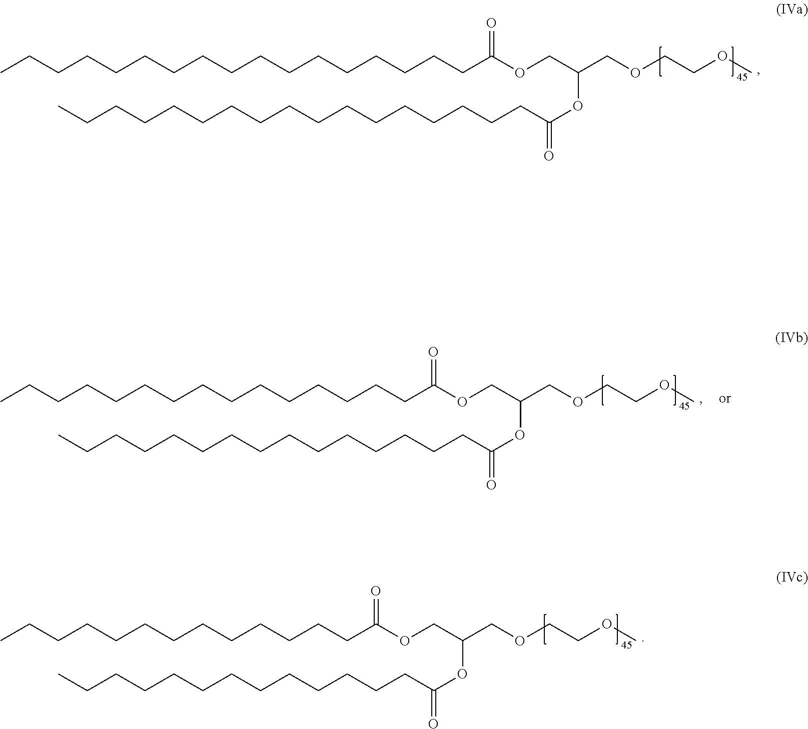

127. The composition according to claim 122, wherein the cationic lipid of formula (I) is selected from:
β-arginyl-2,3-diamino propionic acid-N-palmityl-N-oleyl-amide trihydrochloride 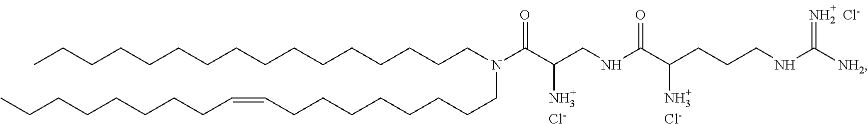 [SMILES: CCCCCCCC/C=C\CCCCCCCCN(CCCCCCCCCCCCCCCC)C(=O)C(N)CNC(=O)C(N)CCCNC(=N)N ; [Cl-] ; [Cl-] ; [Cl-]]
β-arginyl-2,3-diamino propionic acid-N-lauryl-N-myristyl-amide trihydrochloride 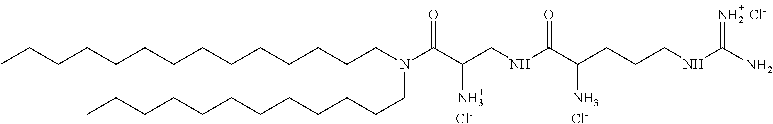 [SMILES: CCCCCCCCCCCCCCN(CCCCCCCCCCCC)C(=O)C(N)CNC(=O)C(N)CCCNC(=N)N ; [Cl-] ; [Cl-] ; [Cl-]]
or
ϵ-arginyl-lysine-N-lauryl-N-myristyl-amide trihydrochloride 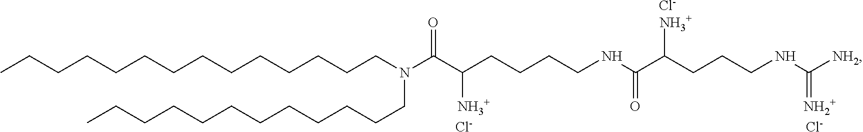 [SMILES: [Cl-] ; CCCCCCCCCCCCCCN(CCCCCCCCCCCC)C(=O)C([NH3+])CCCCNC(=O)C([NH3+])CCCNC(N)=[NH2+] ; [Cl-] ; [Cl-]]
and wherein the sterol compound is selected from cholesterol or stigmasterin; and
wherein the PEGylated lipid is a PEGylated phosphoethanolamine of formula (II) selected from:
1,2-distearoyl-sn-glycero-3-phosphoethanolamine-N-[methoxy(polyethylene glycol)-2000](ammonium salt) 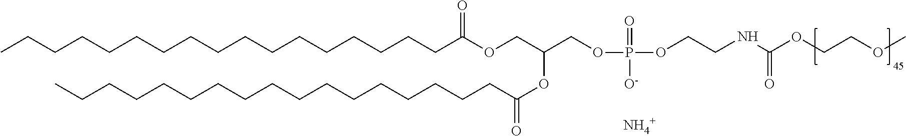
or
1,2-distearoyl-sn-glycero-3-phosphoethanolamine-N-[methoxy(polyethylene glycol)-5000](ammonium salt)

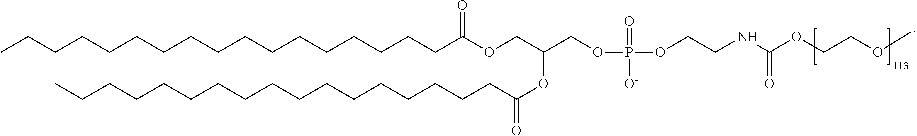

128. The composition according to claim 122, wherein:
the cationic lipid of formula (I) is selected from:
β-arginyl-2,3-diamino propionic acid-N-palmityl-N-oleyl-amide trihydrochloride 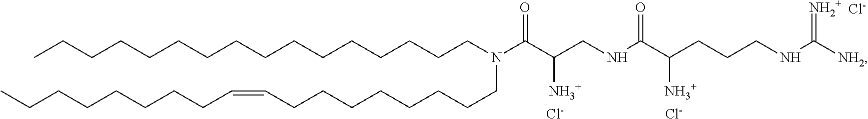 [SMILES: CCCCCCCC/C=C\CCCCCCCCN(CCCCCCCCCCCCCCCC)C(=O)C([NH3+])CNC(=O)C([NH3+])CCCNC(N)=[NH2+] ; [Cl-] ; [Cl-] ; [Cl-]]
β-arginyl-2,3-diamino propionic acid-N-lauryl-N-myristyl-amide trihydrochloride 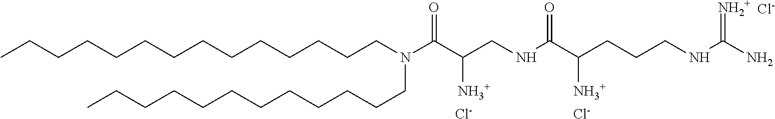 [SMILES: CCCCCCCCCCCCCCN(CCCCCCCCCCCC)C(=O)C([NH3+])CNC(=O)C([NH3+])CCCNC(N)=[NH2+] ; [Cl-] ; [Cl-] ; [Cl-]]
or
ϵ-arginyl-lysine-N-lauryl-N-myristyl-amide trihydrochloride 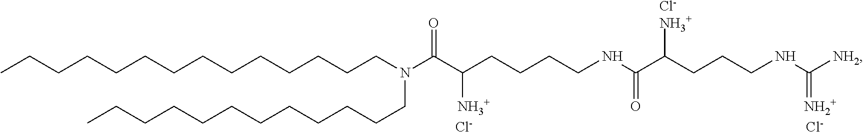 [SMILES: [Cl-] ; CCCCCCCCCCCCCCN(CCCCCCCCCCCC)C(=O)C([NH3+])CCCCNC(=O)C([NH3+])CCCNC(N)=[NH2+] ; [Cl-] ; [Cl-]]
and wherein the sterol compound is selected from cholesterol or stigmasterin; and wherein the PEGylated lipid is a PEGylated ceramide of formula (III) selected from:
N-octanoyl-sphingosine-1-{succinyl[methoxy(polyethylene glycol)2000]} 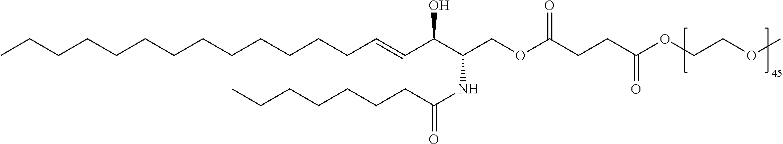
or
N-palmitoyl-sphingosine-1-{succinyl[methoxy(polyethylene glycol)2000]}

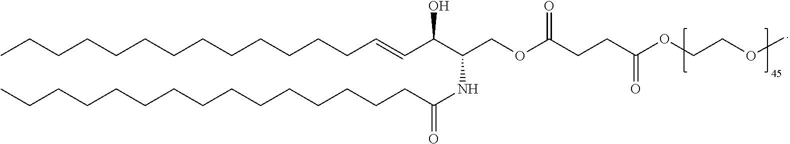

129. The composition according to claim 122, wherein the cationic lipid of formula (I) is selected from:
β-arginyl-2,3-diamino propionic acid-N-palmityl-N-oleyl-amide trihydrochloride 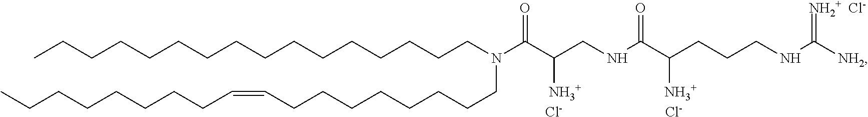 [SMILES: CCCCCCCC/C=C\CCCCCCCCN(CCCCCCCCCCCCCCCC)C(=O)C([NH3+])CNC(=O)C([NH3+])CCCNC(N)=[NH2+] ; [Cl-] ; [Cl-] ; [Cl-]]
β-arginyl-2,3-diamino propionic acid-N-lauryl-N-myristyl-amide trihydrochloride 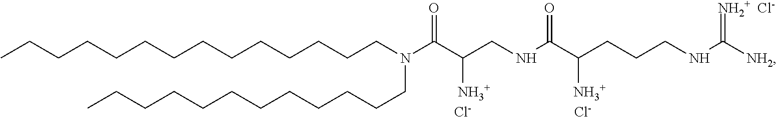 [SMILES: CCCCCCCCCCCCCCN(CCCCCCCCCCCC)C(=O)C([NH3+])CNC(=O)C([NH3+])CCCNC(N)=[NH2+] ; [Cl-] ; [Cl-] ; [Cl-]]
or
ϵ-arginyl-lysine-N-lauryl-N-myristyl-amide trihydrochloride 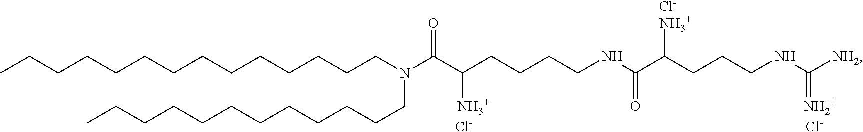
wherein, the sterol compound is selected from cholesterol and stigmasterin; and wherein the PEGylated lipid is a PEGylated diacylglycerol of formula (IV) selected from:
1,2-Distearoyl-sn-glycerol [methoxy(polyethylene glycol)2000] 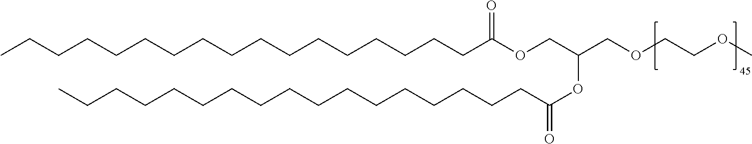
or
1,2-Dipalmitoyl-sn-glycerol [methoxy(polyethylene glycol)2000]

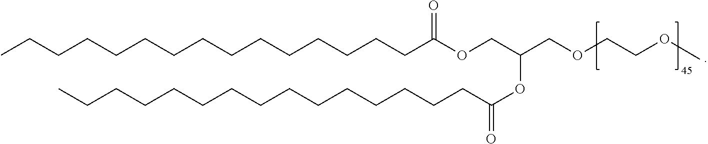

130. The composition according to claim 122, wherein the lipid composition comprises:
70 mole % of β-arginyl-2,3-diamino propionic acid-N-palmityl-N-oleyl-amide trihydrochloride 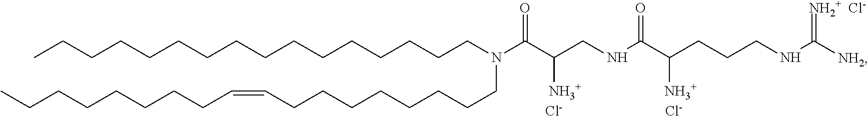 [SMILES: CCCCCCCC/C=C\CCCCCCCCN(CCCCCCCCCCCCCCCC)C(=O)C([NH3+])CNC(=O)C([NH3+])CCCNC(N)=[NH2+] ; [Cl-] ; [Cl-] ; [Cl-]]
29 mole % of cholesterol, and
1 mole % of
1, 2-distearoyl-sn-glycero-3-phosphoethanolamine-N-[methoxy(polyethylene glycol)-2000](ammonium salt)

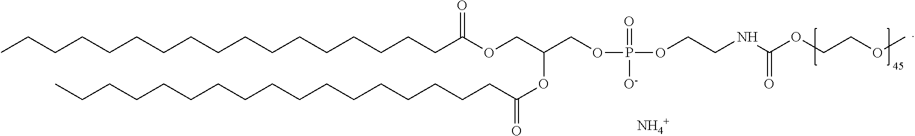

131. The composition according to claim 122, wherein the lipid composition further comprises a 270 mM aqueous sucrose solution.

132. The composition according to claim 122, wherein the composition further comprises a chemical compound, a biologically active agent or a pharmaceutically active agent.

133. The composition according to claim 132, wherein the biologically active agent is an oligonucleotide selected from the group consisting of an siRNA, a microRNA, an siNA, a RNA interference mediating nucleic acid, an antisense nucleic acid, a ribozyme, an aptamer and a spiegelmer.

134. The composition according to claim 133, wherein the ratio between the charged lipid nitrogen atoms to the nucleic acid backbone phosphates (N/P ratio) is from 3 to 12.

135. The composition according to claim 133, wherein the siRNA molecule targets ANG2 gene.

136. The composition according to claim 134, wherein the ANG2 targeting siRNA molecule comprises one or both of the following two sequences:

| | |
|---|---|
| 5′ AgUuGgAaGgAcCaCaUgC 3′ | (SEQ ID NO: 1) |
| and | |
| 5′ gCaUgUgGuCcUuCcAaCu 3′, | (SEQ ID NO: 2) |

and the nucleotides indicated as capital letter are 2′-O-methyl.

137. A method of treating a disease is selected from the group consisting of acute lung injury, acute respiratory distress syndrome, lung cancer, pulmonary metastasis, pulmonary hypertension and pulmonary artery hypertension, comprising the administration of a composition according to claim 136 to a subject having said disease.

138. A pharmaceutical composition comprising a composition according to claim 136 in a therapeutically effective amount and a pharmaceutically acceptable carrier, diluent or excipient.

139. A method for transferring a biologically active compound or a pharmaceutically active compound into a cell or across a membrane of a cell, wherein the method comprises the step of contacting the cell or the membrane of a cell with a composition according to claim 122.

140. A method for the treatment of a disease, wherein the method comprises administering to a subject in need thereof an effective amount of a composition according to claim 132.

141. The method according to claim 140, wherein the composition delivers the chemical compound, the biologically active agent or the pharmaceutically active agent chemical compound into a cell of the subject.
